(19) United States
(12) Patent Application Publication
(10) Pub. No.: US 2024/0026463 A1
(43) Pub. Date: Jan. 25, 2024

(54) EXTRACELLULAR VESICLE PROTEOMIC BIOMARKER PANEL FOR OVARIAN CANCER SCREENING AND THE EARLY DETECTION OF DISEASE

(71) Applicant: University of Kansas, Lawrence, KS (US)

(72) Inventors: Camille V. Trinidad, Kansas City, KS (US); Harsh B. Pathak, Overland Park, KS (US); Mihaela Sardiu, Prairie Village, KS (US); Andrew K. Godwin, Leawood, KS (US)

(21) Appl. No.: 18/356,886

(22) Filed: Jul. 21, 2023

Related U.S. Application Data
(60) Provisional application No. 63/391,657, filed on Jul. 22, 2022.

(51) Int. Cl. C12Q 1/6886

(52) U.S. Cl. CPC C12Q 1/6886; G01N 2800/52; G01N 2800/7028

(57) ABSTRACT

A method of reporting a diagnoses of cancer in a subject is provided. The method can include obtaining a biological sample from the subject, and measuring a presence or amount of a combination of biomarkers in the biological sample. The combination of biomarkers includes ACSL4, IGSF8, ITGA2, ITGA5, ITGB3, and MYOF, and optionally STX4 and/or optionally FOLR1. The presence of the biomarkers in the sample indicates the presence of cancer cells in the subject, and/or an increased amount of the biomarkers in the sample indicates presence of cancer cells in the subject. The method can include determining whether the presence or amount of the combination of biomarkers indicates the presence of cancer cells in the subject, and then preparing a report on the presence of cancer cells in the subject.

### CROSS-REFERENCE TO RELATED APPLICATIONS

This patent application claims priority to U.S. Provisional Application No. 63/391,657 filed Jul. 22, 2022, which provisional is incorporated herein by specific reference in its entirety.

### U.S. GOVERNMENT RIGHTS

This invention was made with government support under GM130423 and CA260132 awarded by the National Institutes of Health. The government has certain rights in the invention.

### BACKGROUND

### Field

The present disclosure relates to protein biomarkers carried by circulating extracellular vesicles (EVs) to support screening of asymptomatic women who are at an increased risk of developing ovarian cancer (e.g., BRCA1 and BRCA2 mutation carriers). The proposed biomarkers are based on proteins found in EVs released from fallopian tube epithelium and maintained in the EVs associated with high grade serous ovarian cancer (HGSOC), i.e., lineage specific markers given this deadliest form of ovarian cancer arise in the fallopian tube. The biomarkers can be the proteins or the mRNA that encodes the proteins.

### Description of Related Art

Over two-thirds of all women diagnosed with epithelial ovarian cancer are likely to die from the disease (>14,000 deaths annually). Even though epithelial ovarian cancer therapies have been researched, little progress has been made in the last few decades. Although the five-year survival rates for most other solid tumors have improved steadily, ovarian cancer remains an exception, making it the deadliest of all gynecological cancers and five times deadlier than breast cancer. Ovarian cancer is hard to detect in its early stages due to its vague symptoms. Women may experience constipation, bloating, pelvic/abdominal pain, trouble eating/feeling full quickly, urgency or frequency of urination. While ovarian cancer tends to occur in post-menopausal women, women at younger ages can be at risk due to genetic predisposition.

Treatment is more effective when diagnosed early, with a five-year survival rate of up to 90%. Unfortunately, most cases are not detected until after the cancer has spread, resulting in a dismal five-year survival rate of less than 30%. Current screening methods for ovarian cancer typically use a combination of a pelvic examination, transvaginal ultrasonography, and serum cancer antigen 125 (CA125), but these have minimal impact on improving mortality. Thus, there is a compelling unmet need to develop new molecular tools that can be used to diagnose early-stage endothelial ovarian cancer and/or assist in the clinical management of the disease after a diagnosis, given that over 220,000 women are living with ovarian cancer in the U.S. and are at risk of recurrence.

High grade serous ovarian carcinoma (HGSOC) accounts for ˜70% of ovarian cancer cases. Recent studies have shown that most HGSOCs are not truly ovarian in origin and often arise from the epithelial cells within the fimbriated end of the fallopian tubes. Molecular studies have suggested that the development from serous tubal intraepithelial carcinoma (STIC) to an adenocarcinoma can take between 6 to 7 years, thus providing a window of opportunity for diagnosis and treatment, such as opportunistic salpingectomy to prevent HGSOCs from developing. Early detection while in the fallopian tube could be essential, given that once the tumor cell leaves the fallopian tube the disease spreads rapidly and is hard to cure.

The foregoing and additional information regarding the background of epithelial ovarian cancer, extracellular vesicles and relation to cancer, detection strategies, and therapies can be found in the incorporated references.¹⁻³⁶

### SUMMARY

In some embodiments, a method of reporting a diagnosis of cancer in a subject is provided. The method can include obtaining a biological sample from the subject and measuring a presence or amount of a combination of biomarkers in the biological sample. The combination of biomarkers includes ITGB3 (CD61), ITGA2 (CD49b), ITGA5 (CD49e), FACL4 (ACSL4), IGSF8 (CD316), and MYOF, and optionally FOLR1 and/or optionally STX4. In some aspects, the presence of the biomarkers in the sample indicates the presence of cancer cells in the subject. In some aspects, an increased amount of the biomarkers in the sample (e.g., compared to a control or sample) indicates the presence of cancer cells in the subject. The method can include determining whether the presence or amount of the combination of biomarkers indicates the presence of cancer cells in the subject and then preparing a report on the presence of cancer cells in the subject. In some aspects, the report includes an association of the combination of biomarkers and the presence of cancer cells. The report is provided to the subject (e.g., including family or caretaker of the subject) or to a medical entity or medical practitioner. In some aspects, the cancer is ovarian cancer or a cancer of fallopian tube origin. In some aspects, the subject is a female human

In some embodiments, a method is provided for detecting a panel of extracellular vesicle-associated protein biomarkers in a female human. The biomarkers can be used for detecting cancer cells, such as those from epithelial ovarian cancer, or any cancer from a fallopian tube tissue origin. The method can include step a) for obtaining extracellular vesicle-associated proteins from a biological sample from a female human. Step b) includes providing a panel of binding agents to the extracellular vesicle-associated proteins, wherein the binding agents are configured to bind with extracellular vesicle-associated proteins of ACSL4, IGSF8, ITGA2, ITGA5, ITGB3, and MYOF and optionally FOLR1, and optionally STX4. Step c) includes assaying for binding a plurality of the binding agents of the panel with the plurality of extracellular vesicle-associated proteins. Step d) includes detecting binding of the plurality of binding agents of the panel with the plurality of extracellular vesicle-associated proteins, the detected plurality of extracellular vesicle-associated proteins being the extracellular vesicle-associated protein biomarkers. Step e) includes preparing a report on the detection of binding of the binding agents to the extracellular vesicle-associated proteins of ACSL4, IGSF8, ITGA2, ITGA5, ITGB3, and MYOF and optionally STX4. Step 0 includes providing the report to the subject or to a medical entity or medical practitioner. These steps can be performed as described herein or otherwise known. In some aspects, the panel of binding agents are designed to be selective for at least ACSL4, IGSF8, ITGA2, ITGA5, ITGB3, and MYOF based on a higher area under curve (AUC) over a negative control. That is, each binding agent is selective for only one of the biomarkers, wherein the selectivity can be suitable for providing the selectivity in accordance with the data provided herein. In some aspects, the panel of binding agents are selective for at least ACSL4, IGSF8, ITGA2, ITGA5, ITGB3, MYOF, and STX4 based on higher area under curve (AUC) over a negative control (e.g., no cancer or non-ovarian cancer cases). In some aspects, the panel of binding agents are selective for at least ACSL4, IGSF8, ITGA2, ITGA5, ITGB3, MYOF, and FOLR1 based on higher area under curve (AUC) over a negative control (e.g., no cancer or non-ovarian cancer cases). In some aspects, the panel of binding agents are selective for at least ACSL4, IGSF8, ITGA2, ITGA5, ITGB3, MYOF, STX4 and FOLR1 based on higher area under curve (AUC) over a negative control (e.g., no cancer or non-ovarian cancer cases).

In some embodiments, the method can further include selecting the female human by at least one of several factors. For example, the female human subject can be suspected of having ovarian cancer or is at an increased life-time risk of developing ovarian cancer, due to carrying an inherited mutations in BRCA1, BRCA2 or other cancer susceptibility genes. Also, the female human subject can be identified as being labeled as being in a risk group for developing ovarian cancer base on family history of breast and/ovarian cancer. Additionally, the female human subject can be identified as being devoid of symptoms of ovarian cancer and these biomarkers can be used to screen average risk women over the age of 55 (or postmenopausal).

In some embodiments, the method described herein can include obtaining and presenting information in the report. The report can be prepared based on the data of the binding of the binding agents with the biomarkers. Accordingly, the report can be prepared, whether manually or by automation, to include one or more of: an identification of the ACSL4, IGSF8, ITGA2, ITGA5, ITGB3, and MYOF and optionally STX4 and optionally FOLR1 as biomarkers of ovarian cancer or cancer of fallopian tube origin; a measurement data that indicates a presence or increased amount of ACSL4, IGSF8, ITGA2, ITGA5, ITGB3, and MYOF, and optionally STX4; a measurement data of control for ACSL4, IGSF8, ITGA2, ITGA5, ITGB3, and MYOF, and optionally STX4; a statement of possible outcomes of ovarian cancer; a statement of treatments for ovarian cancer, wherein the treatments are selected from chemotherapy, radiation therapy, surgical removal of ovarian cancer, or combinations thereof; a statement of possible outcomes of treatments for ovarian cancer; or a listing of medical entities that perform, oversee, or control the treatments for ovarian cancer.

In some embodiments, a panel of binding agents is provided, wherein the binding agents are configured for binding with extracellular vesicle-associated protein biomarkers of ovarian cancer. The binding agent can be any type of binding agent, such as antibodies, antibody fragments, aptamers, binding ligands, combinations thereof, or any molecule that selectively binds with the protein biomarker. In some aspects, the plurality of binding agents are configured to bind to the extracellular vesicle-associated protein biomarkers selectively. In some aspects, the binding agents are configured to selectively bind with a plurality of extracellular vesicle-associated protein biomarkers consisting of ACSL4, IGSF8, ITGA2, ITGA5, ITGB3, and MYOF, and optionally STX4 and optionally FOLR1.

In some aspects, an assay system can include a biochip functionalized for antibody conjugation and at least one composition having a panel of antibody binding agents that bind with the biomarkers.

In some embodiments, a method for treating a female human for ovarian cancer is provided. The method can include a step a) for obtaining extracellular vesicle-associated proteins from a biological sample from the female human. Step b) includes providing a panel of binding agents to the extracellular vesicle-associated proteins. The binding agents are configured to bind with extracellular vesicle-associated proteins of ACSL4, IGSF8, ITGA2, ITGA5, ITGB3, and MYOF, and optionally STX4 and/or optionally FOLR1. Step c) includes assaying for binding a plurality of the binding agents of the panel with the plurality of extracellular vesicle-associated proteins. Step d) includes detecting binding of the plurality of binding agents of the panel with the plurality of extracellular vesicle-associated proteins, where the detected plurality of extracellular vesicle-associated proteins are the extracellular vesicle-associated protein biomarkers. Step e) includes preparing a report on the detection of binding of the binding agents to the extracellular vesicle-associated proteins of ACSL4, IGSF8, ITGA2, ITGA5, ITGB3, and MYOF, and optionally STX4, wherein the report has an identification of the ACSL4, IGSF8, ITGA2, ITGA5, ITGB3, and MYOF and optionally STX4 as biomarkers of ovarian cancer. Step 0 includes providing the subject with a treatment for ovarian cancer, wherein the subject undergoes the treatment for ovarian cancer.

The foregoing summary is illustrative only and is not intended to be in any way limiting. In addition to the illustrative aspects, embodiments, and features described above, further aspects, embodiments, and features will become apparent by referencing the drawings and the following detailed description.

### BRIEF DESCRIPTION OF THE FIGURES

The foregoing and following information as well as other features of this disclosure, will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings.

FIG. 1A shows the representative nanoparticle tracking analysis (NTA) data.

FIG. 1B shows sixty (60) EV particles were imaged, and their size was measured for representative samples by TEM at ×30K magnification.

FIG. 1C shows representative fluorescence data obtained using ExoView for FT and HGSOC tissue explant derived EVs.

FIG. 2A includes a flow chart of the protocol of the analysis pipeline.

FIG. 2B shows the initial number of proteins identified in cell line EVs (blue, average of two or three EV isolations from conditioned media) and tissue explant EVs (gray, average of all samples from their respective groups).

FIG. 2C includes a heatmap of proteomic data showing the enrichment of common EV protein markers for cell line and tissue derived EVs.

FIG. 2D shows a Venn diagram comparison of protein distribution between HGSOC cell lines and tissue explants.

FIG. 2E shows the difference between FT cell lines and FT tissue explants.

FIG. 2F shows the identification of the FT/HGSOC core proteome by comparison of common proteins between the two groups (HGSOC EVs and FT EVs).

FIG. 2G shows the identification of transmembrane proteins within the FT/HGSOC core proteome compared to the SwissProt predicted transmembrane database.

FIG. 2H shows the removal of expected/common EV proteins within the transmembrane FT/HGSOC core proteome compared to the Exocarta and Vesiclepedia.

FIG. 3 shows the detection of predicted transmembrane proteins in FT and HGSOC cell line EVs using capillary western blotting. Capillary western blotting evaluated one antibody per each of the 45 candidate transmembrane proteins.

FIG. 4A includes representative IHC images from the tissue microarrays consisting of 100 patient samples containing benign FT, primary, and metastatic tumor tissue sections are shown for all markers except ITGA2; for ITGA2, tissue samples with higher IHC scores were selected for this figure.

FIG. 4B shows p53-overexpressed STIC and p53-null STIC tissue sections from RRSO. p53 staining was done using an automated Dako Autostainer Link; a manual staining protocol was performed for the other markers.

FIG. 5A shows the quantification of transmembrane exo-protein biomarkers on captured CD81+ EVs (the dotted line signifies background fluorescence from a negative control channel labeled as BKG). FOLR1 was included as a previous positive control for HGSOC³². p-values were calculated using the Mann-Whitney U test.

FIG. 5B shows the area under the curve plot of receiver operating characteristic analyses for all the six markers, and FOLR1 are shown.

The elements and components in the figures can be arranged in accordance with at least one of the embodiments described herein, and which arrangement may be modified in accordance with the disclosure provided herein by one of ordinary skill in the art.

FIGS. 6A1-6A5 are a heatmap of the relative expression levels of the 7 exo-protein biomarkers in 70 cancer samples (10 ovarian cancer and 60 non-ovarian cancers) and 20 healthy controls.

FIG. 6B is a scatter plot showing quantification of 6 transmembrane exo-protein biomarkers on captured CD81+ EVs for 70 cancer samples (10 ovarian cancer and 60 non-ovarian cancers) and 20 healthy controls. FOLR1 was included as a positive control for HGSOC.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to biomarkers for use in early ovarian cancer detection, where the biomarkers can include a panel of extracellular vesicle-associated proteomic biomarkers. The biomarkers can be liquid biopsy biomarkers that are obtained by a liquid biopsy, such as blood. The biomarkers (EV-based or exo-proteins) described herein can be measured for their presence or amount known measurement techniques. The biomarkers can be measured by measuring the presence or amount of the exo-protein biomarker form.

In some embodiments, the biomarker can be a protein cargo that is carried by EVs. The EVs can be considered to be a family including exosomes, small EVs, shedding microvesicles, ectosomes, apoptotic bodies, autophagic EVs, and nanoparticles. All these types of EVs contain various biological molecules. Exosomes and secreted microvesicles carry proteins, lipids, and nucleic acids. The biomarkers of the present invention can be obtained from any of these EVs.

These different types of EVs can shuttle nucleic acids, lipids, and proteins from their cell of origin to surrounding cells to regulate the function of other cells. EVs are classified according to size (from a few nanometers to a few micrometers) and sub-cellular origin. A subtype of EVs, commonly termed exosomes, are endocytic in origin and include 60-80 nm small exosomes (Exo-S) and 90-120 nm large exosomes (Exo-L). In general, the term “exosomes” is broadly used to refer to a heterogenous mixture of small EVs (sEVs) that are less than 200 nm in size; this is because widely used purification methods (such as differential ultracentrifugation) cannot definitively isolate EV class based on sub-cellular origin.

In some embodiments, exo-proteins can be used as biomarkers to detect ovarian cancers, including high grade serious ovarian carcinomas (HGSOCs). The present exo-protein biomarkers for ovarian cancer detection can be used to screen the blood and other bodily fluids of women to determine whether or not the woman is has the earliest stages of disease, even before the manifestation of symptoms. The biomarkers can be detected in a window for those at increased risk for developing ovarian cancer before the onset of the adenocarcinoma phase. The biological sample can be taken as early as possible and processed for the presence of the biomarkers.

In some embodiments, once the biomarkers are detected, the detection and likely outcomes, with or without treatments, can be provided to the subject patient, such as in the form of a report. Treatments to obtain some of the likely outcomes can be identified, and the protocol of the treatment can be provided to the subject (e.g., in the report). Then, subject patient can then determine the desired outcome and whether or not a particular treatment. In one aspect, a treatment can include surgery to specifically remove the fallopian tubes while maintaining the ovaries (and prevent early menopause) can achieve the desired outcome. In one aspect, the treatment can then be performed on the subject to obtain the desired outcome, which can be removal of the ovarian cancer, and possibly other tissues associated with the ovarian cancer, such as the fallopian tubes.

In some embodiments, the biomarkers can be protein biomarkers positioned on the surface of EVs, which are shared between EVs released by fallopian tube (FT) and HGSOC cells. This is important since the discovery represents lineage associated biomarkers, i.e., present in the progenitor cells of HGSOC which are the fallopian tube epithelium. The exo-protein biomarkers described herein have been identified via a comprehensive proteomic analysis of EVs derived from FT and HGSOC cells (both from human tissue samples and established cell lines). These biomarkers have been clinically validated to show that the transmembrane proteins—ACSL4, IGSF8, ITGA2, ITGA5, ITGB3, and MYOF, and optionally STX4—are presented on the surface of EVs from human blood samples. These biomarkers can be used in a panel that can diagnose ovarian cancer in women with high sensitivity and specificity. This allows women to be detected earlier so that the choice of therapy has a higher likelihood of achieving the desired outcome, which is to be cancer free.

ACSL4 (e.g., Acyl-CoA Synthetase Long Chain Family Member 4) is a protein encoded by the ACSL4 coding gene. Other diseases associated with ACSL4 include intellectual developmental disorder, X-Linked 63, Stroke, and Ischemia. Among its related pathways are fatty acid metabolism and integration of energy metabolism. The ACSL4 protein is an isozyme of the long-chain fatty-acid-coenzyme A ligase family.

ACSL4 Table

| Species | Human | Mouse |
|---|---|---|
| Entrez | 2182 | 50790 |
| Ensembl | ENSG00000068366 | ENSMUSG00000031278 |
| UniProt | O60488 | Q9QUJ7 |
| RefSeq (mRNA) | NM_004458 | NM_001033600 |
| | NM_022977 | NM_019477 |
| | NM_001318509 | NM_207625 |
| | NM_001318510 | |
| RefSeq (protein) | NP_001305438 | NP_001028772 |
| | NP_001305439 | NP_062350 |
| | NP_004449 | NP_997508 |
| | NP_075266 | |

Immunoglobulin superfamily member 8 (IGSF8) is a protein that in humans is encoded by the IGSF8 gene. This protein is known to interact with CD81/CD9 complex.

IGSF8 Table

| Species | Human | Mouse |
|---|---|---|
| Entrez | 93185 | 140559 |
| Ensembl | ENSG00000162729 | ENSMUSG00000038034 |
| UniProt | Q969P0 | Q8R366 |
| RefSeq (mRNA) | NM_001206665 | NM_080419 |
| | NM_052868 | |
| | NM_001320247 | |
| RefSeq (protein) | NP_001193594 | NP_536344 |
| | NP_001307176 | |
| | NP_443100 | |

Integrin alpha-2 (ITGA2 or CD49b (cluster of differentiation 49b)) is a protein that in humans is encoded by the CD49b gene. The CD49b protein is an integrin alpha subunit. It makes up half of the a2(31 integrin duplex. Integrins are heterodimeric integral membrane glycoproteins composed of a distinct alpha chain and a common beta chain. They are found on a wide variety of cell types including T cells (the NKT cells), NK cells, fibroblasts and platelets. Integrins are involved in cell adhesion and also participate in cell-surface-mediate signaling. Expression of CD49b in conjunction with LAG-3 has been used to identify type 1 regulatory (Tr1) cells. The DX5 monoclonal antibody recognizes mouse CD49b.

ITGA2 Table

| Species | Human | Mouse |
|---|---|---|
| Entrez | 3673 | 16398 |
| Ensembl | ENSG00000164171 | ENSMUSG00000015533 |
| UniProt | P17301 | Q62469 |
| RefSeq (mRNA) | NM_002203 | NM_008396 |
| RefSeq (protein) | NP_002194 | NP_032422 |

Integrin alpha-5 (ITGA5) is a protein that in humans is encoded by the ITGA5 gene. The ITGAG protein product of this gene belongs to the integrin alpha chain family. Integrins are heterodimeric integral membrane proteins composed of alpha and beta chains. This gene encodes the integrin alpha 5 chain. Alpha chain 5 undergoes post-translational cleavage in the extracellular domain to yield disulfide-linked light and heavy chains that join with beta 1 to form a fibronectin receptor. In addition to adhesion, integrins are known to participate in cell-surface mediated signalling.

ITGA5 Table

| Species | Human | Mouse |
|---|---|---|
| Entrez | 3678 | 16402 |
| Ensembl | ENSG00000161638 | ENSMUSG00000000555 |
| UniProt | P08648 | P11688 |
| RefSeq (mRNA) | NM_002205 | NM_010577 |
| | | NM_001314041 |
| RefSeq (protein) | NP_002196 | NP_001300970 |
| | | NP_034707 |

Integrin beta-3 (β3) (ITGB3 or CD61) is a protein that in humans is encoded by the ITGB3 gene. The ITGB3 protein product is the integrin beta chain beta 3. Integrins are integral cell-surface proteins composed of alpha and beta chains. A given chain may combine with multiple partners, resulting in different integrins. Integrin beta 3 is found along with the alpha IIb chain in platelets. Integrins are known to participate in cell adhesion as well as cell-surface-mediated signaling.

ITGB3 Table

| Species | Human | Mouse |
|---|---|---|
| Entrez | 3690 | 16416 |
| Ensembl | ENSG00000259207 | ENSMUSG00000020689 |
| UniProt | P05106 | O54890 |
| RefSeq (mRNA) | NM_000212 | NM_016780 |
| RefSeq (protein) | NP_000203 | NP_058060 |

Myoferlin (MYOF) is a protein that in humans is encoded by the MYOF gene. The protein encoded by this gene is a type II membrane protein structurally similar to dysferlin. Mutations in dysferlin, a protein associated with the plasma membrane, can cause muscle weakness that affects both proximal and distal muscles. It is a ferlin family member and associates with plasma and nuclear membranes.

MYOF Table

| Species | Human | Mouse |
|---|---|---|
| Entrez | 26509 | 226101 |
| Ensembl | ENSG00000138119 | ENSMUSG00000048612 |
| UniProt | Q9NZM1 | Q69ZN7 |
| RefSeq (mRNA) | NM_013451 | NM_001099634 |
| | NM_133337 | NM_001302140 |
| | | NM_177035 |
| RefSeq (protein) | NP_038479 | NP_001093104 |
| | NP_579899 | NP_001289069 |

Syntaxin-4 (STX4) is a protein that in humans is encoded by the STX4 gene.

STX4 Table

| Species | Human | Mouse |
|---|---|---|
| Entrez | 6810 | 20909 |
| Ensembl | ENSG00000103496 | ENSMUSG00000030805 |
| UniProt | Q12846 | P70452 |
| RefSeq (mRNA) | NM_001272095 | NM_009294 |
| | NM_001272096 | |
| | NM_004604 | |
| RefSeq (protein | NP_001259024 | NP_033320 |
| | NP_001259025 | |
| | NP_004595 | |

Folate receptor 1 (Folate receptor alpha, FOLR1) is a protein that in humans is encoded by the FOLR1 gene. The protein encoded by this gene is a member of the folate receptor (FOLR) family. Members of this family have a high affinity for folic acid and for several reduced folic acid derivatives, and mediate delivery of 5-methyltetrahydrofolate to the interior of cells.

FOLR1 Table

| Species | Human | Mouse |
|---|---|---|
| Entrez | 2348 | 14275 |
| Ensembl | ENSG00000110195 | ENSMUSG00000001827 |
| UniProt | P15328 | P35846 |
| RefSeq (mRNA) | NM_016730 | NM_001252552 |
| | NM_000802 | NM_001252553 |
| | NM_016724 | NM_001252554 |
| | NM_016725 | NM_008034 |
| | NM_016729 | |
| RefSeq (protein) | NP_000793 | NP_001239481 |
| | NP_057936 | NP_001239482 |
| | NP_057937 | NP_001239483 |
| | NP_057941 | NP_032060 |

In some embodiments, the present invention can include measuring and determining an amount of the biomarkers by using reverse-transcription polymerase chain reaction (RT-PCR) or reverse-transcription quantitative polymerase chain reaction (RT-qPCR) to determine the presence or amount of the combination of biomarkers.

Reverse transcription polymerase chain reaction (RT-PCR) combines reverse transcription of RNA into DNA (in this context called complementary DNA or cDNA) and amplification of specific DNA targets using polymerase chain reaction (PCR). It is primarily used to measure the amount of a specific RNA, such as mRNA of the biomarkers. This is achieved by monitoring the amplification reaction using fluorescence, a technique called real-time PCR or quantitative PCR (qPCR). Combined RT-PCR and qPCR (RT-qPCR) are used to analyze gene expression and quantify biomarker mRNA for use as described herein. Accordingly, in some aspects, the biomarker can be the mRNA of each identified exo-protein biomarker.

In some embodiments, the present invention can include measuring the biomarkers by proximity ligation. A proximity ligation assay combines antibody-oligo conjugates, enzymatic ligation, and PCR amplification into a sensitive method for quantitative protein detection from small volumes. Proximity ligation extends the capabilities of traditional immunoassays to include direct detection of proteins, protein interactions, extracellular vesicles, and post translational modifications with high specificity and sensitivity. Protein targets, such as the biomarkers, can be readily detected and localized with single molecule resolution and objectively quantified in unmodified cells and tissues. Utilizing only a few cells, sub-cellular events, even transient or weak interactions, are revealed in situ, and subpopulations of cells can be differentiated. Within hours, results from conventional co-immunoprecipitation and co-localization techniques can be confirmed.

In some embodiments, the biomarkers can be used to detect the cancer cells while confined to or fallopian tube or at any stage of cancer progression. It can be beneficial for the biomarkers to be detected at earlier stages when treatments are much more effective. The early detection can be performed from a sample, such as from a liquid sample (e.g., mucus, secretion, blood, etc.) from the vagina or the subject's blood. The sample can be taken at any time and at any age of the female. The sample can be taken before any cancer cells are disseminated from the fallopian tube and spread to the ovary and/or peroneal cavity. Thus, the biomarkers can be used in a non-invasive blood tests or other liquid biopsies for pre-symptomatic screening and early detection of cancer, such as ovarian cancer or cancer of fallopian tube origin.

The biomarkers can be used for the early detection of serous tubal intraepithelial carcinoma (STIC) lesions confined to the fallopian tubes (the earliest stage of disease) and before the tumor cells disseminate into a woman's peritoneal cavity. The biomarkers can also be used to monitor ovarian cancer disease progression, given that they can detect advanced disease with 100% sensitivity and specificity. The biomarkers can be used for detection of minimal residual ovarian cancer calls even after completion of therapies, resulting in clinical “no evidence of disease’ (NED). Thus, these biomarkers provide a robust panel that can be used to screen for ovarian cancer in various stages of progression and treatment.

In some embodiments, the biomarkers can be validated to ensure that they can be used to screen blood samples from asymptomatic women. The biomarkers can be validated by using positive and negative controls. The positive controls may be samples and/or biomarker data and/or biomarker amounts of women who are at an increased risk of developing ovarian cancer due to inherited mutations in BRCA1 or BRCA2 as well as other ovarian cancer related susceptibility genes. Negative controls can be overtly healthy women at an average lifetime risk of ovarian cancer and/or who omit inherited mutations in BRCA1 or BRCA2 or other ovarian cancer related susceptibility genes.

In some embodiments, the biomarkers can be used to screen for ovarian cancer due to the high specificity and sensitivity of these novel biomarkers for ovarian cancer cells. The present method of detection with this set of protein biomarkers can be adapted to a high throughput system, wherein antibodies targeted against these proteins can be applied on microfluidic devices for direct EV capture and/or as secondary probes to assess the relative level of these markers on circulating EVs. Any possible way of measuring the amount of these exo-protein biomarkers can be employed in the present invention.

In some embodiments, the present invention includes a control panel of the biological markers. The control panel includes at least one composition that includes the biomarkers. For example, a synthetic sample can include a defined amount/concentration of each biomarker in the panel, whether protein or mRNA. In another example, each panel biomarker can be in a synthetic sample at a defined amount/concentration. In another example, a plurality of synthetic samples can include the biomarkers in a gradient of the amount/concentration, whether alone or in biomarker combinations. The synthetic samples can be used in the protocols herein as positive controls, where measurements can be done via multiple types of platforms and methodologies. Accordingly, the synthetic samples can be used for positive controls of the biomarkers associated with ovarian cancer disease that has developed in the fallopian tube. In some aspects, the synthetic samples can include the extracellular vesicle associated with the biomarker proteins.

In some embodiments, EVs for liquid-based testing can be purified from a biological sample, such as blood, plasma, vaginal fluid, fallopian tissue or secretion, or other bodily fluids. The purification can be done by size-exclusion chromatography and immunoaffinity capture. The extracellular vesicle-associated biomarkers co-localized on the EV surface can be detected by any means, such as with binding agents or proximity ligation qPCR. For example, antibody combinations comprising one capture antibody and two oligonucleotide-tagged detection antibodies can be used recognize a plurality of unique biomarkers.

EVs can be isolated in some embodiments using size-exclusion chromatography and immunoaffinity capture. The biomarkers co-localized on individual EVs' surface can be detected with proximity ligation qPCR. Using this approach, antibody combinations recognizing a plurality of biomarkers are employed. Each combination for each biomarker consists of one capture antibody and two oligonucleotide-tagged detection antibodies.

In some embodiments, plasma samples from women with early stage I/II high-grade serous ovarian carcinoma can be tested, and the data thereof can be used as positive controls to determine the absolute sensitivity and specificity of these exo-protein biomarkers for earlier forms of the disease. Samples from healthy women without cancer can be used as negative controls.

In some embodiments, the protocols described herein may be applied to other extracellular proteins, such as those from serum.

In some embodiments, a method is provided for detection of a panel of EV-associated protein biomarkers in a female human. The method can include a step of obtaining EV-associated proteins from a biological sample from the female human. Also, a panel of binding agents are provided, which binding agents bind to biomarkers of a biomarker panel that is associated with ovarian cancer. The binding agents are then provided to the obtained EV-associated proteins. The binding agents are configured to bind with the individual EV-associated proteins ACSL4, IGSF8, ITGA2, ITGA5, ITGB3, MYOF, FOLR1 and/or STX4. The binding of the plurality of the binding agents with the panel with the plurality of EV-associated proteins can be assayed for so that the binding of the plurality of binding agents with the panel of biomarkers detected. This detection of binding of the binding agents with the biomarkers can show that the biomarkers are present in the obtained EV-associated proteins from the subject. In some aspects, the amount of the biomarkers is determined, which can be compared to a positive control, a negative control, or a threshold standard. The detected plurality of EV-associated proteins biomarkers are the EV-associated protein biomarkers. The presence or amount of the detection of the biomarkers can then be the basis of generating a report to inform the subject of the detection of the biomarkers and the indication of ovarian cancer or cancer of origin from the fallopian tubes. The report may also include prognosis information, such as stage of cancer and possible progression steps, as well as treatments and outcomes thereof.

In some embodiments, the binding agents are selected from antibodies, antibody fragments, aptamers, or combinations thereof. Each binding agent is configured to bind with one of the protein biomarkers of ACSL4, IGSF8, ITGA2, ITGA5, ITGB3, MYOF, FOLR1, and/or STX4. As such, there is a binding agent for each biomarker. However, the type of binding agent can vary for each biomarker. For example, one biomarker may have an antibody binding agent, but another biomarker can have an aptamer binding agent. Thus, a collection of binging agents can include various binding agent types so long as each binging agent binds one of the biomarkers. In some aspects, the binding agents are antibodies or antibody fragments.

In some embodiments, the panel of binding agents are selective for at least six of the plurality of EV-associated proteins. In some aspects, the panel of binding agents are selective for at least ACSL4, IGSF8, ITGA2, ITGA5, ITGB3, and MYOF, and optionally FOLR1. In some aspects, the panel of binding agents are selective for at least ACSL4, IGSF8, ITGA2, ITGA5, ITGB3, MYOF, and STX4, and optionally, FOLR1. In some aspects, the panel of binding agents are selective for at least ACSL4, IGSF8, ITGA2, ITGA5, ITGB3, MYOF, FOLR1, and STX4. In some aspects, the panel of binding agents are selective for at least ACSL4, IGSF8, ITGA2, ITGA5, ITGB3, MYOF, FOLR1, and optionally STX4.

The methods described herein can include use of tools and protocols for obtaining EVs from the biological sample. The tools and protocols can be configured for measuring the EV-associated proteins from the intact or lysed extracellular vesicles. The methods can include obtaining a biological tissue sample from the female human. The EV-associated proteins can be linked with serous tubal intraepithelial carcinoma (STIC). Any tool or method can be used for obtaining a biological fluid sample from the female human, wherein the biological sample is blood, plasma, urine, and/or a fluid from a vagina of the female human subject.

In some embodiments, the methods can include assaying for binding of a binding agent to the respective biomarker. Such assaying can include a protein binding assay where each binding agent binds with a respective EV-associated protein biomarker from the plurality of EV-associated proteins that are defined as biomarkers for ovarian cancer. The protein binding assay can be configured as an antibody screening assay, wherein each antibody is a binding agent for a respective EV-associated protein biomarker. In some aspects, the antibody screening assay is a simple Western assay. In some aspects, the antibody screening assay can be performed on an assay device that includes a biochip or microfluidic chip configured for receiving the binding agents. However, any type of device and any type of protocol that can be used to detect binding between the binding agents and the biomarkers can be used. For example, the chip can be an ExoProfile chip.

The methods include obtaining EVs from the biological sample, and separating the extracellular vesicle-associated proteins from the extracellular vesicles. The biological sample can be any biological fluid, such as saliva or blood, which can be obtained by any method. The separation can be by any separation technique that can separate the EVs from the biological sample.

In some embodiments, the method of assaying for the biomarkers can include quantifying levels of the EV-associated protein biomarkers by using a reporter. For example, the methods can be performed using fluorescence as a reporter. That is, the fluorescence can be used as a visual indicator of binding between the biomarkers and the binding agents. Various types of fluorescent agents and protocols can be used to track the binding by fluorescence. In some aspects, binding agents (e.g., antibodies) conjugated to biotin are used as a fluorescent reporter, and observing fluorescence of a EV can show the presence of the biomarkers. In some aspects, the assaying is an immunocapture and fluorescent detection assay.

In some embodiments, the biomarker is determined to be present at an elevated level that is greater than a negative control or a threshold. The negative control can be a biomarker panel of no ovarian cancer, or the negative control can be the expression level of the defined biomarkers in one or more non-ovarian cancer patients or an average or mean value thereof, which can be used as a threshold. The levels of the biomarkers are higher than negative controls in subjects that have or are susceptible to ovarian cancer, or at some point of progression of the ovarian cancer.

Also, positive controls can be used, which are biomarker levels in one or more ovarian cancer patients or average or mean thereof. The test values can be compared to the positive control values for correlation of the biomarkers so that the biomarker levels that are similar or about the same as the positive controls are likely to show that the subject has ovarian cancer or some stage of progression thereof.

In some embodiments, the methods can include creating a proteomic profile of EV-associated proteins in the subject, in a negative control, and a positive control. The proteomic profile can include the EV-associated protein biomarkers for the female human that are defined herein as well as others. The profiles can be used for comparison with obtained values to determine if the biomarker is showing as being overexpressed in the subject. This allows for when all the biomarkers being overexpressed the female subject being identified as susceptible or having some stage of progression of ovarian cancer.

In some embodiments, the female human can be selected at random or based on some criteria of related to ovarian cancer. In some aspects, the female human is suspected of having ovarian cancer or is positive for BRCA1 mutation or BRCA2 mutation or other cancer susceptibility genes contributing to an increase lifetime risk of developing this disease. In some aspects, the female is over a certain age, such as over 40, 45, 50, 55, 60, 65, 70, or 75. In some aspects, the female human is in a risk group for developing ovarian cancer. In some aspects, the female human is devoid of symptoms of ovarian cancer.

In some embodiments, a panel of binding agents for EV-associated protein biomarkers of ovarian cancer is provided. In one example, the panel includes binding agents to the EV-associated protein biomarkers, wherein the binding agents are configured to bind with a plurality to biomarkers selected from ACSL4, IGSF8, ITGA2, ITGA5, ITGB3, MYOF, FOLR1, and/or STX4. In one example, the panel includes binding agents to the EV-associated protein biomarkers, wherein the binding agents are configured to bind with a plurality of biomarkers selected from ACSL4, IGSF8, ITGA2, ITGA5, ITGB3, FOLR1, and MYOF. The binding agents can be complementary nucleic acids that bind with mRNA of these biomarkers. In one example, the panel includes binding agents to the EV-associated protein biomarkers, wherein the binding agents are configured to bind with a plurality to biomarkers selected from ACSL4, IGSF8, ITGA2, ITGA5, ITGB3, MYOF, FOLR1, and STX4.

In some embodiments, an assay system is provided. The assay system can include a biochip functionalized for capturing a binding agent that binds with one of the biomarkers. For example, the binding agent can be an antibody, and the biochip is functionalized for antibody conjugation. The system can also include at least one composition having a plurality of binding agents that are configured to bind with a plurality of extracellular protein biomarkers are selected from ACSL4, IGSF8, ITGA2, ITGA5, ITGB3, MYOF, FOLR1, and/or STX4. In some aspects, the biochip includes an anti-CD81 antibody or related tetraspanins, including CD9 and CD63. In some aspects, the plurality of extracellular protein biomarkers are selected from ACSL4, IGSF8, ITGA2, ITGA5, ITGB3, and MYOF. In some aspects, the plurality of extracellular protein biomarkers are selected from ACSL4, IGSF8, ITGA2, ITGA5, ITGB3, FOLR1, and MYOF. In some aspects, the plurality of extracellular protein biomarkers are selected from ACSL4, IGSF8, ITGA2, ITGA5, ITGB3, FOLR1, MYOF, and STX4.

In some embodiments, an assay system can include a PCR or qPCR machine and the required compositions for performing RT-PCR or RT-qPCR.

In some embodiments, the methods can include proteomic analyses by mass spectrometry of EVs.

In some embodiments, the biomarkers exhibit high area under the curve (AUC) values ranging from about 0.85 to about 0.98 as calculated using receiver operating characteristic (ROC) analysis. The AUC range of about 0.85 to about 0.98 can be used as a positive control value, where patient samples in this range likely have ovarian cancer. The biomarkers can have a biomarker AUC threshold of at least 0.85, where an AUC of at least 0.85 indicates the biomarker is present and can indicate cancer. When each panel of the combination of biomarkers has an AUC value greater than the AUC threshold value of there is evidence that the subject has an onset of cancer, such as ovarian cancer or cancer of fallopian tube origin. When multi-marker analysis was performed, the combination of IGSF8 and ITGA5 yielded most significant degree of sensitivity and specificity in this cohort. In some aspects, the threshold can be 0.7 or 0.75 for the AUC, and an AUC above the threshold for the biomarker indicates the presence of the biomarker that indicates the ovarian cancer.

The comparison of the AUC of a female subject with the positive control AUC can provide information about whether or not the female subject, when lower than the positive control the female is likely does not have ovarian cancer, but when about the same value as the positive control the female is likely to have ovarian cancer.

The AUC for a negative control non-ovarian cancer patient is about 0.4-0.6 (0.5 is a random classifier as compared to 1.0 which is a biomarker with perfect performance). The comparison of the AUC of a female subject with the negative control AUC can provide information about whether or not the female subject, when higher than the negative control the female is likely to have ovarian cancer, but when about the same value as the negative control the female is unlikely to have ovarian cancer.

Once the biomarkers have been identified, quantified, and/or compared to controls or standards, the report identifying whether or not the subject has cancer can be prepared. The report can be prepared as any standard report as a paper or electronic file. The report can have information or other indicia that identifies the subject and the presence or absence of cancer. The report can be prepared by typing or writing on paper, or by inputting and word processing on a computing system. As a result, the report provides the information about the subject and cancer and may include information about the biomarkers and their relationship to cancer. In some aspects, the AUCs of the biomarkers of the subject can be provided, which can be shown by comparison to AUC values for the positive controls, negative controls, or threshold values. The report is prepared by an action that takes the information of the determination of the presence of the biomarkers and/or the indication of a potential ovarian cancer and puts the information of the determination into indicia (e.g., text, charts, tables, graphs, images, etc.) that can be read by a human visually or by a computer electronically.

In some embodiments, the report includes one or more of: an identification of the ACSL4, IGSF8, ITGA2, ITGA5, ITGB3, and MYOF and optionally STX4 and/or FOLR1 as biomarkers of cancer of ovaries or fallopian tube origin; a measurement data that indicates the presence or increased amount of ACSL4, IGSF8, ITGA2, ITGA5, ITGB3, and MYOF and optionally STX4 and/or FOLR1; a measurement data of positive or negative control for ACSL4, IGSF8, ITGA2, ITGA5, ITGB3, and MYOF and optionally STX4 and/or FOLR1, or a threshold thereof; a statement of possible outcomes of cancer of ovaries or fallopian tube origin; a statement of treatments for the identified cancer, wherein the treatments are selected from chemotherapy, radiation therapy, surgical removal of the cancer cells or tumor, or combinations thereof; a statement of possible outcomes of treatments for the identified cancer; or a listing of medical entities that perform, oversee, or control the treatments for the identified cancer.

In some embodiments, a method for treating a female human for ovarian cancer is provided. The method can include determining the presence of the combination of biomarkers described herein in the subject, preparing a report that the female human has cancer, and then treating the cancer. The determination of the presence of the biomarkers can be by any method, such as follows. Once the biomarkers that indicate cancer are detected, such as having a AUC over the threshold value or over the negative control value or about the positive control value, the report is prepared. The method can include preparing a report on the detection of binding of the binding agents to the extracellular vesicle-associated proteins of ACSL4, IGSF8, ITGA2, ITGA5, ITGB3, and MYOF and optionally STX4, and optionally FOLR1. The report has an identification of the ACSL4, IGSF8, ITGA2, ITGA5, ITGB3, and MYOF and optionally STX4 and optionally FOLR1 as biomarkers of ovarian cancer. The method includes providing the subject to get a treatment for the ovarian cancer, wherein the subject undergoes the treatment, e.g., surveillance or surgery for early stage ovarian cancer.

In some embodiments, the biomarkers described herein can be used for monitoring the patient during the performance of the treatment for ovarian cancer (e.g., measure minimal residual disease after completion of treatment which is an essential measure of early recurrence and subsequent death). That is, the subject can be treated for cancer, and the biomarkers can be measured. An indication that the subject still has cancer can cause further treatments for cancer therapy to be provided to the subject. An indication that the subject is free of cancer can terminate any ongoing cancer therapy treatment and then monitor the patient. The treatments for ovarian cancer can be selected from chemotherapy, radiation therapy, surgical removal of the fallopian tubes (if the cancer is detected while confined) or the ovarian cancer (if tumor cells have spread to the ovaries and peritoneum), or combinations thereof. In some aspects, the method includes the subject undergoing the treatment of ovarian cancer. In some aspects, the subject can be provided a statement of possible outcomes of treatments for ovarian cancer, or provided a listing of medical entities that perform, oversee, or control the treatments for ovarian cancer.

In some embodiments, the subject is selected by selecting the female human by at least one of: to be suspected of having ovarian cancer or is positive for BRCA1, BRCA2 mutation or other cancer susceptibility genes; is in a risk group for developing ovarian cancer; or is devoid of symptoms of ovarian cancer. Any of these types of subjects can be identified as a subject of the present invention.

In some embodiments, after the subject undergoes the treatment for ovarian cancer, the method can include performing another biomarker detection assay (e.g., measuring CA-125 concentration in serum), and preparing a second report on the detection of binding of the binding agents to the extracellular vesicle-associated proteins of ACSL4, IGSF8, ITGA2, ITGA5, ITGB3, and MYOF and optionally STX4 and/or FOLR1. This new report can be to the subject or to a medical entity or medical practitioner. The method can include performing a subsequent treatment if the second report provides an indication of ovarian cancer in the subject based on the extracellular vesicle-associated proteins of ACSL4, IGSF8, ITGA2, ITGA5, ITGB3, and MYOF and optionally STX4 and/or FOLR1.

### Examples

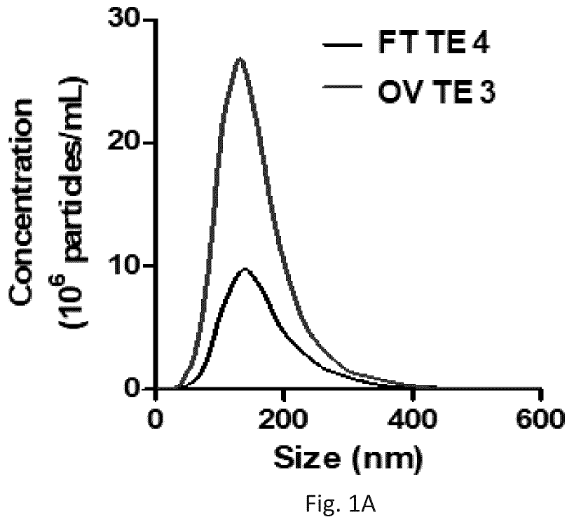

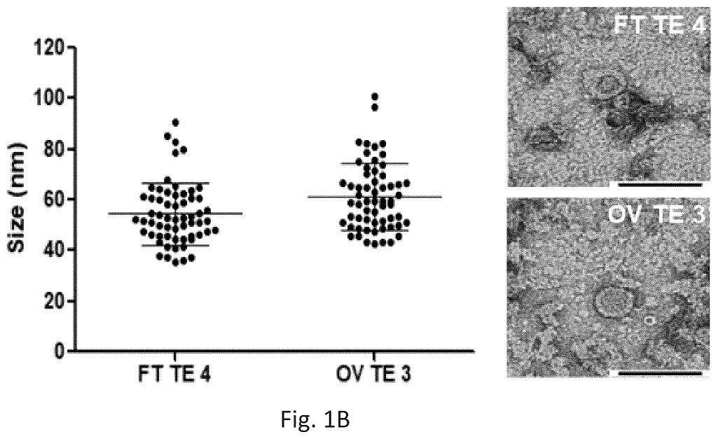

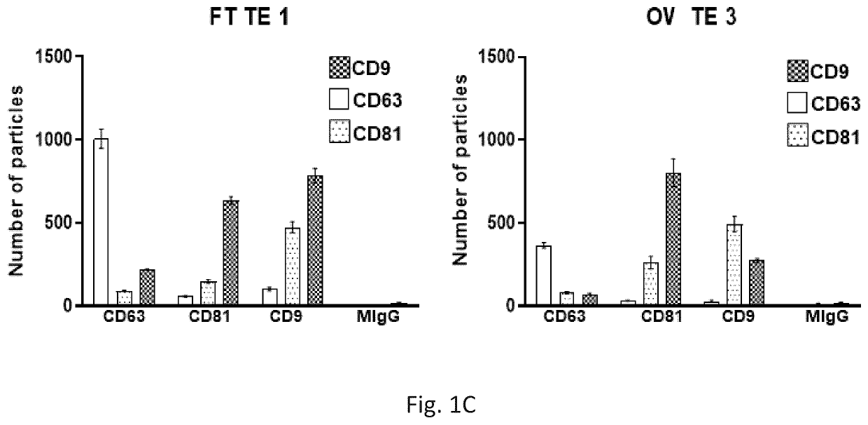

Enrichment and characterization of EVs derived from patient HGSOC and healthy fallopian tube tissue explants and cell lines was performed. Conditioned media was collected from 3 FT cell lines (FT240, FT246 and FT282), 6 HGSOC cell lines (OVCAR2, OVCAR3, OVCAR4, OVCAR8, PEO1 and PEO4) and 21 fresh tissue explants (HGSOC primary tumor tissues, n=9, HGSOC omental metastases, n=6, and healthy FT tissue specimens, n=6). For these studies, the 24 h time point was used for tissue media collection since the number of total particles decreased by at least 55% within 48 h. This observation may be attributed to the decrease in growth factors once the media is replaced at 24 h. The collected media was then processed by differential ultracentrifugation to enrich for the EVs.²⁷ Representative EVs purified from either cell lines or tissue explants were characterized by nanoparticle tracking analysis (NTA), transmission electron microscopy (TEM), single particle interferometric reflectance imaging sensing (SP-IRIS) and fluorescence following the Minimum Information for Studies of Extracellular Vesicles 2018 guidelines (FIGS. 1A-1C).³⁵ By NTA, most particles were between 120-160 nm in size (FIG. 1A). Using negative staining followed by TEM, the EVs where shown to have the typical cup shaped morphology with a size range between 32-128 nm (FIG. 1B). By SP-IRIS, the mode size for the EVs is 50 nm (data not shown). The variation in EV particle size as detected by NTA, TEM, and SP-IRIS and fluorescence is likely due to the differences in instrument sensitivities and their limitations (FIGS. 1A-1B). With SP-IRIS and fluorescence, it was found that both cell line and tissue explant EVs either from FT or HGSOC expressed common tetraspanin markers, e.g., CD9, CD63 and CD81 (FIG. 1C). Also, it was observed that tissue explant derived EVs had a higher percentage of CD63 single positive EVs compared to cell line derived EVs (FIG. 1C) whereas FT cell lines and HGSOC cell lines displayed a higher CD9⁺ population, as well as double positive CD9⁺CD81⁺ EV populations compared to the tissue explant derived EVs (FIG. 1C).

Surgical resections of healthy FT or tumor tissues were minced and used to initiate short-term tissue explants (cultured for 24 h) followed by collection of the conditioned media and processed by differential ultracentrifugation to enrich for EVs. Likewise, conditioned media from the FT and ovarian cancer cell lines shown was collected and processed. FIG. 1A shows the representative NTA data. FIG. 1B shows sixty (60) EV particles were imaged, and their size was measured for representative samples by TEM at ×30K magnification. The mean size with s.e.m. is indicated by the error bars. FIG. 1C shows representative fluorescence data obtained using ExoView for FT and HGSOC tissue explant derived EVs. The EVs were captured using commonly expressed EV tetraspanins, namely, CD9, CD63 and CD81 and probed with detection antibodies conjugated to Alexa Flour dyes: CD9-AF488 (blue), CD63-AF647 (pink) and CD81-AF555 (green). The error bars represent the mean particle count with s.e.m.

Identification of FT and HGSOC core proteome biomarkers was performed. After EV characterization, we performed proteomic profiling and an analysis pipeline to establish the EV proteome of FT and HGSOC (for both tissue and cell lines) via liquid chromatography tandem mass spectrometry (LC-MS/MS), with the goal of identifying putative transmembrane exo-proteins that can ultimately be used to perform immunocapture and detection of intact EVs from clinical samples.

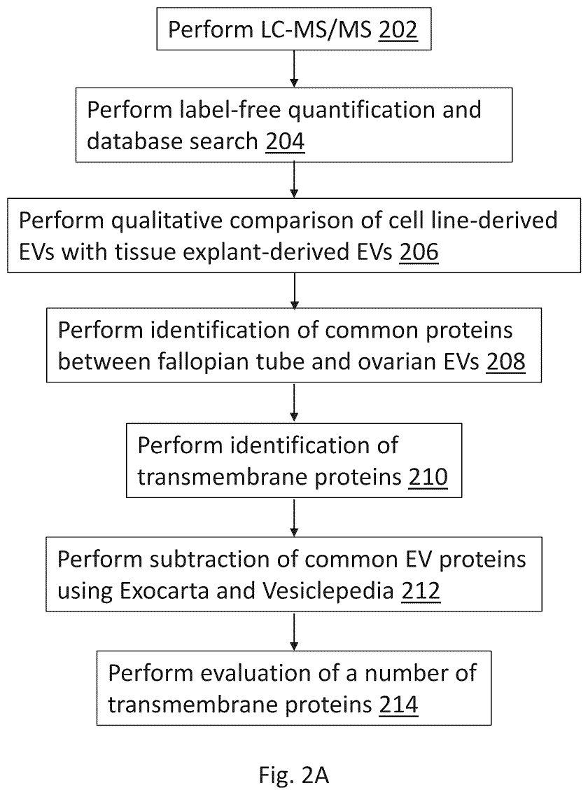

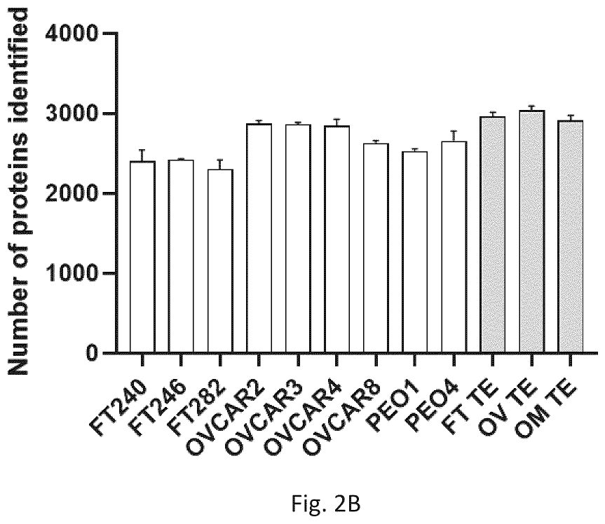

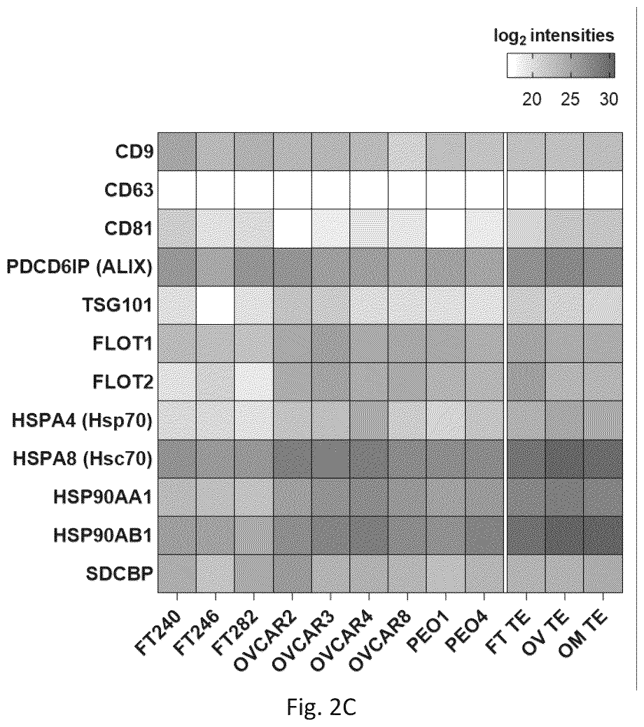

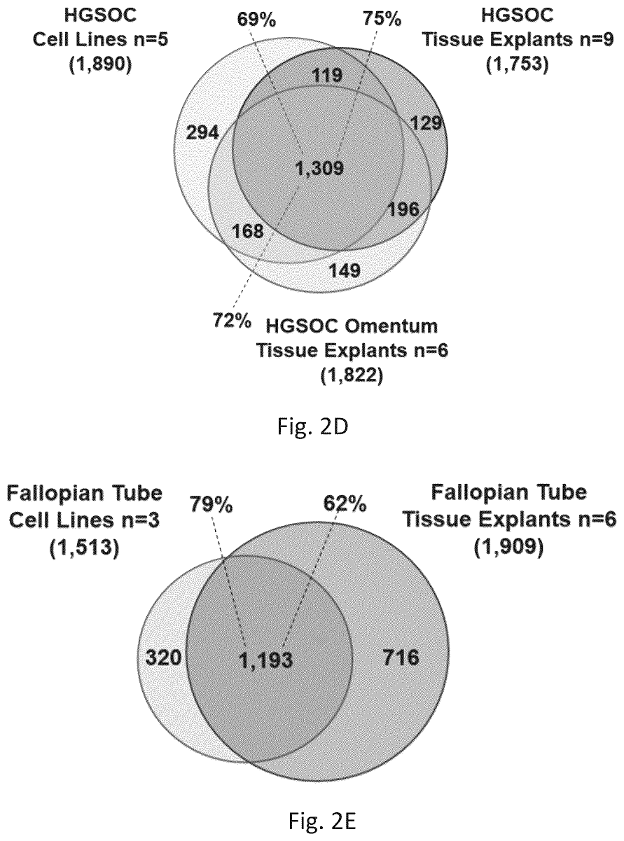

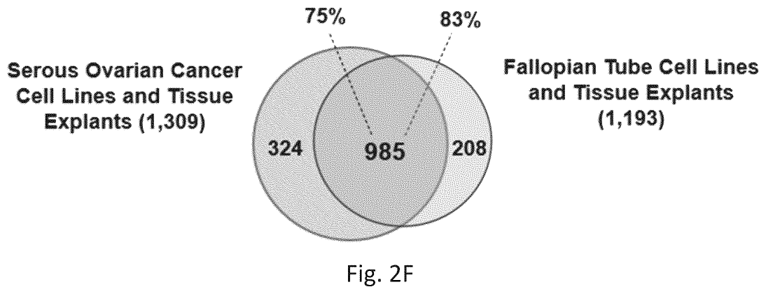

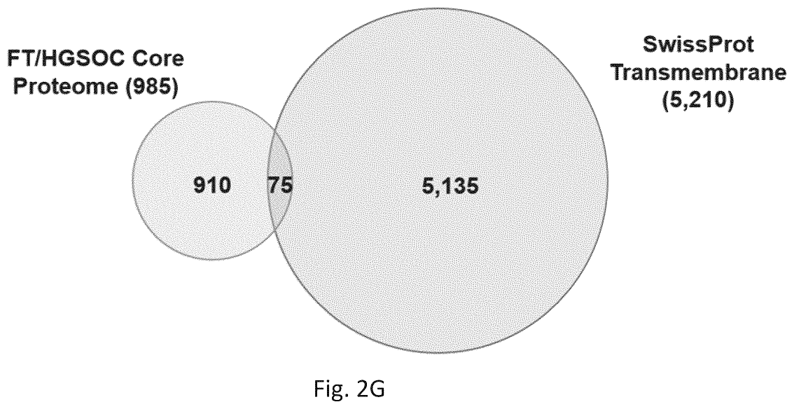

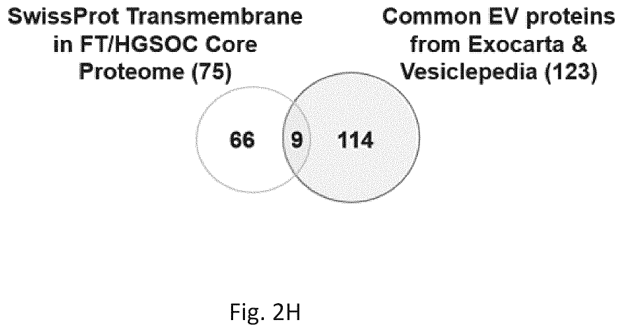

FIG. 2A includes a flow chart of the protocol of the analysis pipeline. The analysis can include performing LC-MS/MS^{27,34} at block 202. The label-free quantification and database search can be performed at block 204. The identification of common proteins between fallopian tube and ovarian EVs can be performed at block 208. The identification of transmembrane proteins can be performed at block 210. The subtraction of common EV proteins can be performed, such as with Exocarta, Vesiclepedia, or other technique to remove common EV proteins (block 212). The evaluation of a number of the transmembrane proteins to be ovarian biomarkers can be performed on the regained EV-associated proteins to determine the biomarkers at block 214.^{10, 17, 37-41}

Approximately 2,200 to 3,200 exo-proteins were identified in each sample (FIG. 2B). The relative abundance of the common EV markers was calculated using proteomic data and it found that most of these markers were identified at similar levels in both the cell line and tissue explant derived EVs (FIG. 2C). However, a common EV marker, CD63, was detected at relatively low levels or was undetectable in EVs, as observed in previous studies²⁷. Lack of representation in the mass spectrometry data is likely due to the CD63 being heavily glycosylated³⁷. As mentioned earlier, it was found that CD63+ EVs was predominately present in tissue explants derived EVs using SP-IRIS and fluorescence. The findings also support a recent study that proposed syntenin-1 (SDCBP) to be a putative universal EV marker³⁸ as this protein was detected at relatively similar levels across all the EV samples (both tissue and cell line-derived). In addition, the study examined the relative quantitation of serum-based proteins that are reported in literature or included in biomarker-based algorithms for ovarian cancer, i.e., CA-125³⁹; ROCA multimodal screening^{10,17}; the multivariate ROMA⁴⁰ test; and the FDA approved OVA1⁴¹ test.

After this initial assessment of the data quality by comparing levels of the canonical EV markers as well as presence of existing serum markers from the tests mentioned above, the data was filtered furthered by comparing the EV proteins from tissue explants with their respective cell lines (e.g., HGSOC or FT) to increase the specificity of the EV proteins to their site of origin (FIGS. 2D-2E). The 1,309 proteins found in the HGSOC group were compared with the 1,193 proteins found in the FT group to identify 985 EV proteins that are common between the two groups which we termed as FT/HGSOC core proteome (FIG. 2F). Extensive ROC analysis of the 985 core FT/HGSOC proteome identified a list of 43 monotone and non-monotone markers 42 (the definition of each marker type is provided in the Material and Methods section), including non-transmembrane and cytosolic proteins as well as a bioinformatic analysis of the 985 core proteins and 324 markers unique to HGSOC (FIG. 2F).

The ROC analysis is useful for identifying differences between FT and HGSOC samples. However, this study can identify 1) lineage-associated markers, i.e., exo-protein biomarkers present in FT epithelium that are preserved in HGSOC, and 2) exo-protein biomarkers that would be suitable for both immunocapture and on chip detection using our microfluidic ExoProfile chip. For this analysis, the 985 FT/HGSOC core proteome with known or predicted transmembrane proteins curated in the protein sequence database of UniProtKB (Swiss-Prot, July 2021), which resulted in a truncated list of 75 exo-proteins (FIG. 2G). Common EV proteins were subtracted by comparing to the list of top 100 EV-associated proteins found in ExoCarta and Vesiclepedia databases. This narrowed the list to 66 transmembrane exo-proteins not commonly observed in EVs (FIG. 2H). The fold differences were calculated for these 66 proteins in the FT- and HGSOC-derived EVs and selected those that showed a log₂ fold-change ≥−0.58 to identify proteins present in the FT samples and which increase in expression as the disease progresses to HGSOC. This approach resulted in a ranked list of 47 exo-proteins. Several of these proteins are integrins which have been implicated in cancer cell proliferation, migration and invasion⁴³. Integrins have also been shown to be essential for EV homing and act as seeds that condition the favorable formation of tumor niches^{24,44}. In addition, two of these proteins, IGHM and ADAM10, were found to be common EV proteins reported in literature^{34,35,45}, which were not previously in listed in the ExoCarta and Vesiclepedia databases so they were manually removed from further analysis.

FIG. 2A shows the pipeline for filtering LC-MS/MS data to aide in selection of potential transmembrane candidate protein biomarkers. FIG. 2B shows the initial number of proteins identified in cell line EVs (blue, average of two or three EV isolations from conditioned media) and tissue explant EVs (gray, average of all samples from their respective groups). FIG. 2C includes a heatmap of proteomic data showing enrichment of common EV protein markers for both cell line and tissue derived EVs. FIG. 2D shows a Venn diagram comparison of protein distribution between HGSOC cell lines and tissue explants, and FIG. 2E between FT cell lines and FT tissue explants. FIG. 2F shows the identification of the FT/HGSOC core proteome by comparison of common proteins between the two groups (HGSOC EVs and FT EVs). FIG. 2G shows the identification of transmembrane proteins within the FT/HGSOC core proteome by comparison to the SwissProt predicted transmembrane database. FIG. 2H shows the removal of expected/common EV proteins within the transmembrane FT/HGSOC core proteome by comparison to the Exocarta and Vesiclepedia.

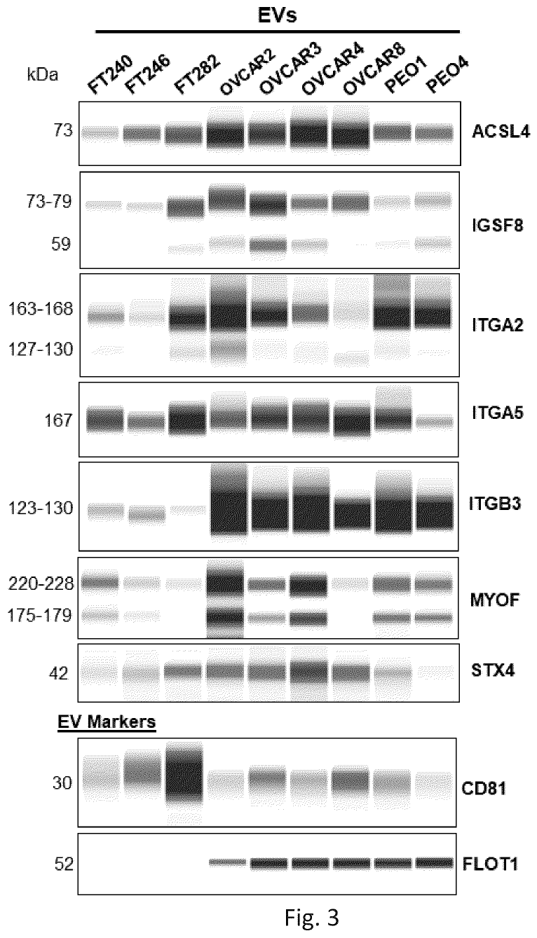

Capillary western blotting (Simple Western—using the Wes platform) was performed on the 45 transmembrane proteins using one antibody per protein to confirm their presence within cell line derived EVs to support the LC-MS/MS data. Among the proteins analyzed using Wes, ACSL4, IGSF8, ITGA2, ITGA5, ITGB3, MYOF and STX4 were consistently detected in all the tested FT and HGSOC cell line EVs (FIG. 3). These identified exo-protein biomarkers were prioritized for further analysis.

FIG. 3 shows the detection of predicted transmembrane proteins in FT and HGSOC cell line EVs using capillary western blotting. One antibody per each of the 45 candidate transmembrane protein was evaluated by capillary western blotting. The detected exo-proteins, confirmed to be present in EVs from 6 HGSOC and 3 FT cell lines are shown. Exo-proteins that did not meet the criteria of being present in EVs from all tested FT and HGSOC cell lines are not included in this figure. In addition, CD81 and FLOT1 were evaluated as these are common EV markers.

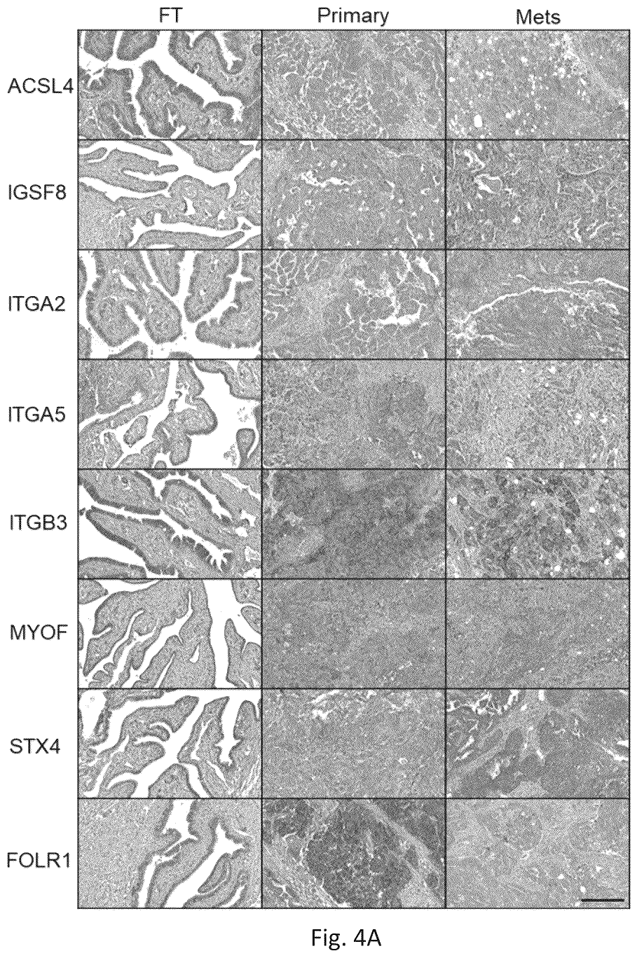

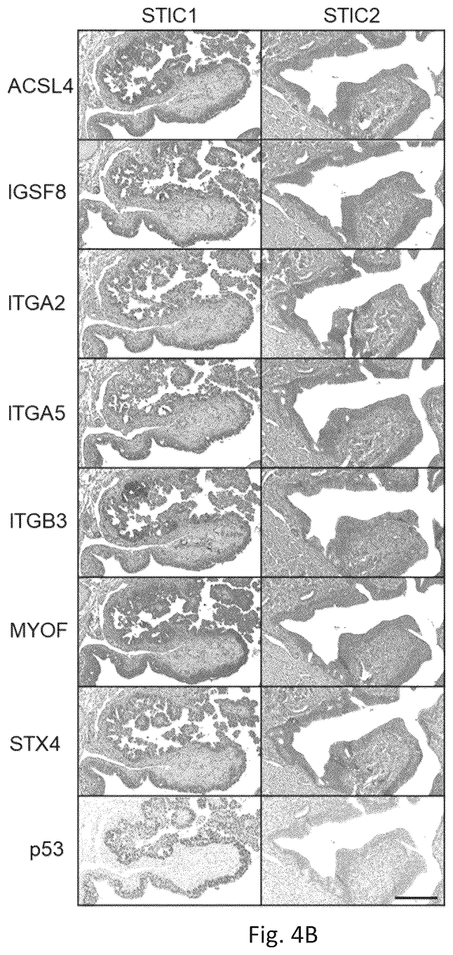

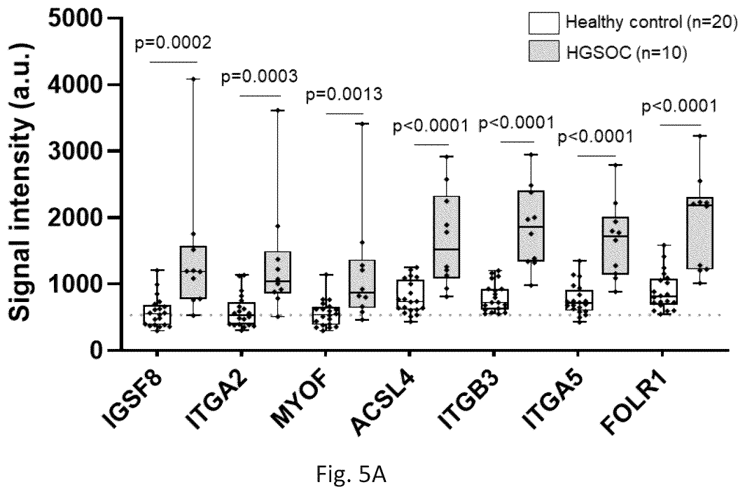

The expression of these proteins was measured via immunohistochemistry (IHC) to confirm the tissues of origin. We created a tissue microarray (TMA) using samples from 100 patients with most of the samples having matching primary tumor, metastatic tumor, and a healthy region of fallopian tube tissue. Following staining, a pathologist reviewed and scored each core (FIG. 4A). It was found that all transmembrane proteins were expressed to varying degrees in healthy FT tissue, and in both primary and metastatic tumors. FOLR1 is used as a positive control since it has been shown to be highly expressed in ovarian tumors compared to healthy tissue and is decreased in platinum-resistant ovarian tumors compared to drug-sensitive tumors^{46,47}. ITGA2 showed lower expression in all tissues compared to all the other proteins. It was found that in both p53-overexpressed and p53-null STICs, most of the candidate biomarker proteins were expressed in these regions. ITGA2 was found to be only expressed in the p53-null STIC, while ITGB3 and ITGA5 demonstrated patches of staining within STIC lesions (FIG. 4B). The IHC protein expression results show that these transmembrane proteins are present in the fallopian tube tissue and are maintained during disease progression.

FIGS. 4A-4B show immunohistochemistry staining of tissues from patients with HGSOC and FT tissue with STICs showing expression of the candidate transmembrane proteins. FIG. 4A includes representative IHC images from the tissue microarrays consisting of 100 patient samples containing benign FT, primary, and metastatic tumor tissue sections are shown for all markers except ITGA2; for ITGA2, tissue samples with higher IHC scores were selected for this figure. FIG. 4B shows p53-overexpressed STIC and p53-null STIC tissue sections from RRSO. p53 staining was done using an automated Dako Autostainer Link; a manual staining protocol was performed for the other markers. The scale bars represent 200 μm. Macrosections of tissues that included HGSOC, kidney, liver, placenta, spleen and tonsil were used as negative and positive controls. These macrosections of tissues were also in the optimizing the antibody concentrations.

After confirming that these transmembrane proteins are present in tissues via IHC, the exo-proteins in patient plasma samples were tested using a modified ExoProfile microfluidic chip capable of EV immunocapture and fluorescence detection³². To further improve the capacity of this device for molecular profiling of circulating EVs, a platform can integrate an ultrasensitive gold nanorod (AuNR) plasmonic fluor-linked immunosorbent assay (P-FLISA) with the ExoProfile chip. In contrast to conventional ELISA that uses reporter enzymes for signal amplification, which limits the multiplicity, our assay uses gold nanorods coated with fluorophore molecules as the ultrabright fluorescent tag for the sandwich immunoassay. Due to the localized surface plasmon resonance of AuNRs, fluorescence signal can be enhanced by more than 1000-fold with immensely improved stability. Furthermore, this new platform enables sensitive capture and detection of EVs without the additional steps needed for in-solution enzymatic reaction and thus greatly simplifies the assay workflow and expedites the chip operation and analysis speed.

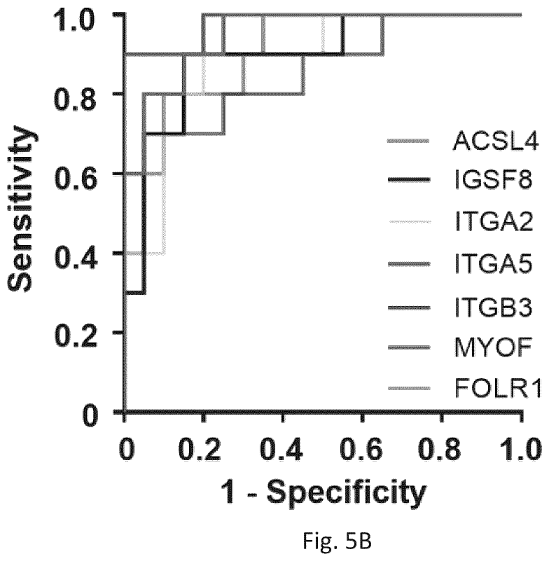

The biomarker exo-FOLR1 was able to differentiate ovarian patients (late and early stage) from benign and healthy controls with high specificity and sensitivity^{32,48}. Therefore, FOLR1 was used as the positive control to assess the specificity and sensitivity of the current lineage specific biomarkers being evaluated. Using the ExoProfile chip specifically developed to simultaneously measure multiple EV-associated analytes, the protocol captured CD81+ EVs from plasma (n=10 cases and n=20 age/race matched controls) and quantified the relative levels of ACSL4, IGSF8, ITGA2, ITGA5, ITGB3, MYOF, and FOLR1 (FIG. 5A). The plasma samples were obtained from HGSOC patients with both early (FIGO Stage I-II; n=5) and advanced stage (FIGO Stage III-IV, n=5) disease. To ensure the quality of the sample set, we also quantified serum CA-125 and plasma EV concentration using ELISA. Notably, all seven FT/HGSOC exo-protein biomarkers were detected at significantly higher levels in the HGSOC plasma (regardless of stages) relative to healthy plasma having p-values ≤0.001. ROC analysis was performed for each marker and found that four markers (ACSL4, ITGB3, ITGA5 and FOLR1) had an area under the curve (AUC) that is higher for early stage compared to healthy plasma versus late stage compared to healthy plasma. The protocol included calculating all the ovarian cancer patients (n=10) together instead of in separate groups (early or late stage) against the healthy controls (n=20). It was found that these markers had AUCs ranging from 0.85 to 0.98 (FIG. 5B and Table 1), which indicates a high separation between diseased and healthy controls. In fact, exo-ITGA5 and exo-ITGB3 (AUC of 0.95 and 0.98, respectively) performed better than exo-FOLR1 (AUC of 0.925) which was a robust ovarian cancer biomarker³². Various marker combinations of two or more markers were studied, and it was found that the linear combination of IGSF8 and ITGA5 based on logistic regression analysis yielded an AUC of 0.990 with a sensitivity of 0.80 at 99.8% specificity (Table 1). Based on multivariable logistic regression analysis, the equation was derived for the best marker combination is as follows: Linear combination of IGSF8 & ITGA5=11.299×log(IGSF8)+14.935×log(ITGA5). These results confirm that the lineage associated exo-protein biomarkers detected from proteomic profiling of FT/HGSOC tissue-derived EVs can be incorporated onto the ExoProfile chip to develop a clinically relevant liquid biopsy test focused on the early detection of this disease, and potentially while confined to the fallopian tubes.

TABLE 1
Receiver operating characteristic tests to calculate area
under the curve for each exo-protein marker were performed.

| | | | | | | Sensitivity |
|---|---|---|---|---|---|---|
| | | Standard | | | | at 99.8% |
| Exo-Protein | AUC | Error | CI_lower | CI_upper | p-value-1t | Specificity |
| IGSF8 | 0.895 | 0.064 | 0.770 | 1.000 | 2.77 × 10⁻⁴ | 0.300 |
| ITGA2 | 0.885 | 0.064 | 0.759 | 1.000 | 3.82 × 10⁻⁴ | 0.400 |
| MYOF | 0.850 | 0.080 | 0.692 | 1.000 | 1.12 × 10⁻³ | 0.400 |
| ACSL4 | 0.915 | 0.052 | 0.813 | 1.000 | 1.42 × 10⁻⁴ | 0.600 |
| ITGB3 | 0.980 | 0.022 | 0.938 | 1.000 | 1.33 × 10⁻⁴ | 0.900 |
| ITGA5 | 0.950 | 0.037 | 0.878 | 1.000 | 4.12 × 10⁻⁵ | 0.600 |
| FOLR1 | 0.925 | 0.047 | 0.832 | 1.000 | 1.01 × 10⁻⁴ | 0.600 |
| Linear | 0.990 | 0.020 | 0.964 | 1.000 | 1.33 × 10⁻⁵ | 0.800 |
| Combination | | | | | | |
| of IGSF8 & | | | | | | |
| ITGA5 | | | | | | |

TABLE 2
Area under the curve values for early and late stage
ovarian cancer patient (OC) samples compared to
healthy controls (HC) on the ExoProfile chip

| Exo- | HC vs Early Stage OC | HC vs Late Stage OC |
|---|---|---|
| Protein | AUC | AUC |
| IGSF8 | 0.820 | 0.970 |
| ITGA2 | 0.830 | 0.940 |
| MYOF | 0.770 | 0.930 |
| ACSL4 | 0.920 | 0.910 |
| ITGB3 | 1.000 | 0.960 |
| ITGA5 | 0.970 | 0.930 |
| FOLR1 | 0.970 | 0.880 |

FIGS. 5A-5B show the evaluation of transmembrane exo-proteins in plasma samples using ExoProfile chips. FIG. 5A shows quantification of transmembrane exo-protein biomarkers on captured CD81+ EVs (dotted line signifies background fluorescence from a negative control channel labeled as BKG). FOLR1 was included as a previous positive control for HGSOC³². The p-values were calculated using the Mann-Whitney U test. FIG. 5B shows the area under the curve plot of receiver operating characteristic analyses for all the six markers and FOLR1 are shown.

When the protein biomarkers are used with the functionalized ExoProfile chip, the protocol can distinguish the HGSOC patients (both early and late stage equally) from matched healthy individuals with high AUC values. Furthermore, when combined IGSF8 and ITGA5 the protocol achieved a sensitivity of 0.80 at 99.8% specificity. These results exceed the performance of clinically adopted serum multimarker panels, i.e., CA-125 (gold standard) and HE4^{61,62}. Although this study was performed on a small set of cases and control, future studies will need to validate the sensitivity and specificity of these exo-biomarkers in clinical samples from other gynecological and non-gynecological malignancies, and ultimately asymptomatic-high risk women who subsequently are diagnosis of HGSOC.

### Material and Methods

### Human Samples

De-identified plasma samples from healthy and untreated HGSOC patients with early (FIGO Stage I-II) or advanced stage (FIGO Stage III-IV) disease were obtained from the University of Kansas Medical Center Biospecimen Repository Core Facility (KUMC BRCF).

Primary HGSOCs tumors or metastatic tissue were obtained from women with Stage II-IV HGSOCs who were undergoing tumor debulking surgery. Healthy FT tissues were obtained from patients undergoing salpingo-oophorectomy for various medical conditions, including hysterectomies for non-cancerous conditions. Archival formalin-fixed paraffin-embedded (FFPE) FT tissue samples with STIC lesions were obtained from women undergoing RRSO after pathological review. Informed consent was obtained from all participants included in the study. The de-identified tissues obtained were minced into small pieces and placed in 6-well plates containing 3 mL of cell media. Cell media without supplements and without serum was added and placed in a humidified incubator at 37° C. with 5% CO₂ for 24 h. The conditioned media was collected and EVs were enriched from the media using differential ultracentrifugation as described below.

### Cell Culture

FT cell lines, FT240, FT246 and FT282⁶³ were a kind gift from Dr. Ronny Drapkin (University of Pennsylvania). All cell lines were validated by short tandem repeat fingerprinting. FT cell lines were cultured in a 50/50 mixture of DMEM/F-12 without L-glutamine (Corning) supplemented with 2% (v/v) Ultroser G (Pall Biosciences) and 1% (v/v) penicillin-streptomycin at 37° C. with 5% CO₂. Ultroser G was ultracentrifuged for a minimum of 18 h at 100,000×g followed by filtration through a 0.2 μm filter. Ovarian cancer cell lines (OVCAR2, OVCAR3, OVCAR4, OVCAR8, PEO1 and PEO4) were cultured in RPMI1640 media (Hyclone, Cytiva Life Sciences) supplemented with 10% (v/v) EV-depleted FBS (spun at 100,000×g at 4° C. for at least 18 h and filtered using a μm filter), 2.5% mg/mL insulin and 100 units/mL penicillin-streptomycin at 37° C. with 5% CO₂. Conditioned media was collected when cells were at least 60% confluent. Conditioned media from each cell line was collected from either two separate passages (FT cells) or three separate passages (for HGSOC cells). Each collection was processed and maintained as an independent biological replicate even throughout the LC-MS/MS procedure and analysis.

Enrichment of EVs from Conditioned Media by Differential Ultracentrifugation

Conditioned media from cell cultures and tissue explants was collected and centrifuged at 300×g for 10 min to remove cell debris. The supernatant fraction was then centrifuged at 2,000×g for 20 min to remove apoptotic bodies. This was followed with supernatant centrifugation at 10,000×g to remove large microvesicles for 1 h followed by 100,000×g spin to collect EVs. The EV pellets were washed and resuspended in PBS and spun for 1 h at 100,000×g. The EV pellets were resuspended in PBS and stored at −80° C.

### Transmission Electron Microscopy

Glow-discharged carbon-coated copper grids were floated on the surface of a drop of 30 μL of EVs for 20 min. The grids were then rinsed with water followed by negative staining with 1% uranyl acetate for 5 s. Once the grids were dry, TEM images were taken using a JEM-1400 Transmission Electron Microscope (JEOL USA, Inc.) equipped with a Lab6 gun.

### Nanoparticle Tracking Analysis (NTA)

The concentration and size of the enriched EVs were analyzed using the NanoSight LM10 instrument (Malvern Panalytical Ltd). NTA was performed using a monochromatic 404 nm laser on EVs diluted in 0.2 μm filtered PBS. Three recordings of 60 s videos were taken per sample at camera level 13 using the NTA software version 2.3. Data was compiled using a custom MATLAB code.

### Single Particle Interferometric Reflectance Imaging Sensing and Fluorescence

Enriched EV samples (17.5 μL each) were mixed with an equal volume of Solution A. The sample was then placed on an ExoView chip and incubated overnight. 1 mL of Solution A was added, and this was shaken at 500 rpm for 3 min at room temperature. The chip was then washed three-times with an incubation solution before adding the blocking solution containing tetraspanin antibodies (CD9, CD63 and CD81). The chips were incubated for 1 h at room temperature. Incubation solution was added and shaking at 500 rpm for 3 min at room temperature was repeated. The ExoView chip was washed 3 times before adding rinse solution and was scanned using the ExoView R100 instrument (NanoView Biosciences). nScan 2.8.10 software was used for data acquisition and NanoViewer software was used for data analysis (both from NanoView Biosciences). The threshold used for cut-off was 500 arbitrary units of fluorescence for the red, green, and blue channels in all experiments.

### Mass Spectrometry

Enriched EV samples were submitted to the Proteomics and Mass Spectrometry Facility at the Donald Danforth Plant Science Center (St. Louis, MO) for LC-MS/MS analysis. Twenty micrograms of EVs per sample were denatured using 8 M urea, reduced with 10 mM TCEP, and alkylated with 25 mM iodoacetamide followed by digestion with trypsin/Lys-C mix at 37° C. overnight. The digested sample was acidified with 1% TFA and then cleaned up using a Pierce C18 tip (Thermo Fisher Scientific). The extracted peptides were dried down and each sample was resuspended in 30 μL 1% acetonitrile/1% formic acid. Approximately 1 μg of each sample was injected for LC-MS/MS analysis.

LC-MS/MS was carried out on an Orbitrap Fusion Lumos (Thermo Fisher Scientific) mass spectrometer coupled with a U3000 RSLCnano HPLC (Thermo Fisher Scientific). The peptide separation was carried out on a C18 column (Acclaim PepMap RSLC, 50 cm×75 μm nanoViper™, C18, 2 μm, 100 Å, Thermo Fisher Scientific) at a flow rate of 0.3 μL/min and the following gradient: Time=0-4 min, 2% B isocratic; 4-8 min, 2-10% B; 8-83 min, 10-25% B; 83-97 min, 25-50% B; 97-105 min, 50-98%. Mobile phase A consisted of 0.1% formic acid and mobile phase B consisted of 0.1% formic acid in acetonitrile. The instrument was operated in the data-dependent acquisition mode in which each MS1 scan was followed by higher-energy collisional dissociation (HCD) of as many precursor ions in a 2-second cycle (Top Speed method). The mass range for the MS1 done using the FTMS was 365 to 1800 m/z with resolving power set to 60,000 @ 400 m/z and the automatic gain control (AGC) target set to 1,000,000 ions with a maximum fill time of 100 ms. The selected precursors were fragmented in the ion trap using an isolation window of 1.5 m/z, an AGC target value of 10,000 ions, a maximum fill time of 100 ms, a normalized collision energy of 35 and activation time of 30 ms. Dynamic exclusion was performed with a repeat count of 1, exclusion duration of 30 s, and a minimum MS ion count for triggering MS/MS set to 5000 counts.

### Identification and Label Free Quantification of Proteins

Sequence mapping and label-free quantification were done using Proteome

Discoverer (PD) version 2.4 (Thermo Fisher Scientific). Database searches with Sequest search engine were launched in PD and queried against Human reference proteome (Uniprot.org, April 2021). The digestion enzyme was set as trypsin. The MS/MS spectra were searched with a precursor mass tolerance of 10 ppm and a fragment ion mass tolerance of 0.6 Da. Carbamidomethylation of cysteine was set as a fixed modification. Oxidation of methionine and acetylation of the N-terminal of protein were specified as variable modifications. Matched peptides were filtered using a Percolator-based 1% false discovery rate (FDR). Protein quantification was achieved by using the total intensities of all precursors.

### Simple Western Assay (Wes)

The concentration of EV proteins was established using the Bradford assay (Bio-Rad) according to the manufacturer's instructions. Simple Western assay (Wes, ProteinSimple) was used for the detection of EV markers (CD81 and Flotillin-1) and proteins that were selected for further evaluation, namely ACSL4, IGSF8, ITGA2, ITGA5, ITGB3, MYOF and STX4. EVs at a concentration of 0.4 μg/μL were used in these assays. The 12-230 kDa or 66-440 kDa Wes separation module and the secondary anti-mouse, anti-rabbit, and anti-goat detection modules were used following manufacturer's instructions. The chemiluminescent detection profile was set at High Dynamic Range 4.0 and contrast was manually adjusted for each sample. Data were analyzed using the Compass software version 6.0.0. (ProteinSimple).

TABLE 1
Antibodies used in this study.

| Antibodies | Manufacturer | Catalog # | Dilution | Application |
|---|---|---|---|---|
| ACSL4 | Novus Biologicals | NBP2-16401 | 1:50 | Wes |
| | | | 1:200 | IHC |
| ACSLA | Novus Biologicals | NB300-861 | 1:500 | ExoProfile chip |
| CA-125 | Abcam | ab274402 | NA | ELISA |
| CD81 | Proteintech | 66866-1-Ig | 1:100 | Wes |
| CD81 | Ancell | 302-820 | 1:5 | ExoProfile chip |
| Flotillin-1 | Santa Cruz | sc-74566 | 1:50 | Wes |
| FOLR1 | R&D systems | AF5646 | 1:500 | ExoProfile chip |
| FOLR1 | Invitrogen | PA5-24186 | 1:400 | IHC |
| IGSF8 | Novus Biologicals | AF3117 | 1:20 | Wes |
| IGSF8 | R&D systems | MAB31171-100 | 1:100 | ExoProfile chip |
| IGSF8 | Invitrogen | PA5-52875 | 1:100 | IHC |
| ITGA2 | Novus Biologicals | NBP2-76483 | 1:20 | Wes |
| | | | 1:200 | IHC |
| ITGA2 | Novus Biologicals | NBP3-03851 | 1:100 | ExoProfile chip |
| ITGA5 | Novus Biologicals | NBP1-84576 | 1:50 | Wes |
| | | | 1:200 | IHC |
| ITGA5 | R&D systems | AF1864 | 1:500 | ExoProfile chip |
| ITGB3 | Novus Biologicals | NBP2-67416 | 1:50 | Wes |
| | | | 1:50 | IHC |
| ITGB3 | R&D systems | AF2266 | 1:500 | ExoProfile chip |
| MYOF | Novus Biologicals | NBP1-84694 | 1:20 | Wes |
| | | | 1:100 | ExoProfile chip |
| | | | 1:200 | IHC |
| p53 | Dako | IR616 | NA | IHC |
| STX4 | Novus Biologicals | MAB7894-SP | 1:20 | Wes |
| STX4 | Invitrogen | PA5-51560 | 1:300 | IHC |
| Donkey anti-Goat | Novus Biologicals | NBP1-74820 | 1:500 | ExoProfile chip |
| IgG (H + L) secondary | | | | |
| antibody (biotin) | | | | |
| Donkey anti-Rabbit | Novus Biologicals | NBP1-75288 | 1:500 | ExoProfile chip |
| IgG (H + L) secondary | | | | |
| antibody (biotin) | | | | |

### Immunohistochemistry Staining of TMA and STICs

Unstained tissue slide sections of five TMA blocks containing representative tissue cores of benign fallopian tube, primary and metastatic ovarian tumor tissues (n=100) were provided by the KUMC BRCF. Tissue microarrays were previously constructed by the BRCF staff using archival FFPE tissue blocks and were provided as a kind gift by Dr. Dineo Khabele (Washington University, St. Louis). Tissue sections were placed in xylene and rehydrated in ethanol baths of decreasing concentration. Antigen retrieval was performed by heating with citrate buffer in a pressure cooker for 15 minutes. Once at room temperature, endogenous peroxidase activity was blocked using a BLOXALL blocking solution (Vector Laboratories) for 20 min, slides were washed followed by another blocking step using 2.5% normal horse serum for 30 min. Primary antibody incubation using ACSL4, IGSF8, ITGA2, ITGA5, ITGB3, MYOF, STX4 and FOLR1 was performed overnight. Anti-mouse, anti-rabbit, or anti-goat secondary ImmPRESS horse IgG polymer reagent (Vector Laboratories) was added and incubated for 30 min. The ImmPACT DAB EqV reagent was then incubated for 1-5 min. A light hematoxylin counterstain was performed followed by dehydration, clearing, and mounting using a permanent mounting medium (Vector Laboratories). These stained slides were then visualized under a bright field microscope and scored by a board-certified pathologist using the following formula: H-score=(0×area of cells with absent staining)+(1×area of “1+” cells %)+(2×area “2+” cells %)+(3×area “3+” cells %)⁶⁴.

FT tissue sections containing STIC lesions were stained for p53 using an automated protocol optimized for the Dako Autostainer Link (Agilent) or for the candidate protein biomarkers using the manual IHC staining protocol described above.

### EV Immunoassay Using the ExoProfile Chip

The ExoProfile chip was functionalized for antibody conjugation and an anti-CD81 antibody was flowed through the chip to coat the surface similar to previous methods³². De-identified plasma samples, first processed to remove platelets (2,500×g for 15 min), were centrifuged at 10,000×g for 30 min at 4° C. to remove large microvesicles. 10 μL of plasma was diluted to 100 μL and used to detect seven exo-protein biomarkers (ACSL4, IGSF8, ITGA2, ITGA5, ITGB3, FOLR1 and MYOF) simultaneously. One channel was designated as the negative control (PBS) to measure any background fluorescence. 10 μL of diluted plasma flowed through the chip with a constant flow rate of 5 μL/h. The chip was washed with Superblock buffer and primary detection antibodies against the target exo-proteins were added using the optimized concentrations. Biotin-conjugated secondary antibodies (donkey anti-goat IgG or donkey anti-rabbit IgG) were flowed through followed by the plasmonic fluorophores to serve as the fluorescence reporter^{65,66}. Fluorescence images were captured using a Nikon Ti2 inverted fluorescence microscope equipped with a LED excitation light source (Lumencor). Fluorescence intensity was quantified by processing and analyzing the images using ImageJ.

### Bioinformatics Analysis

Qualitative analysis (GO identification and network analysis) of the FT/HGSOC core proteome was analyzed using DAVID version 6.8. The protein-protein interaction network was obtained using STRING version 11.5. p-values were calculated using EdgeR analysis⁶⁷. The log₂ fold change values and the adjusted p-values were used to define significant differential expression proteins. Qprot⁶⁸ v1.3.5 was used to calculate Z-statistics, log₂ (fold change) and FDR values. Qprot analysis was performed with a burn-in value of 2,000 and 10,000 iterations.

To identify monotone and non-monotone markers, we used the length of the ROC curve⁴². The length of the ROC curve for each protein was estimated non-parametrically using a Gaussian kernel density estimator for the scores of each group (FT and HGSOC), so that a smooth ROC curve estimate is available. Monotone markers are proteins which have an ROC curve length of 2 and exhibit AUC>0.8 and AUC<0.2, while non-monotone markers are proteins that have an ROC curve length of >1.6 and exhibit AUC≥0.35 but ≤0.65.

### Statistical Analysis

For NTA data and IHC expression score analysis, GraphPad Prism version 8 was used for performing Mann-Whitney U tests in calculating p-values to compare between the samples. Data were expressed as means±s.e.m. (standard error of the mean). For the ExoProfile chip, one-way ANOVA was performed on the fluorescent intensities measured per each sample per biomarker. ROC/AUC for single marker analysis was performed using GraphPad Prism version 8. For combination marker analysis, we explored all possible combinations of markers which involved scrutinizing all possible models with 2 markers, with 3 markers etc., along with a model that included all markers. The Akaike Information Criterion (AIC) was used to identify the best marker combination⁶⁹. The linear term of this logistic regression model is then extracted to be utilized as the combined marker score. We then maximized the Youden index⁷⁰ to derive the sensitivity and specificity at the optimized cutoff.

### Selectivity

Data has been obtained that show that lineage specific (FT/HGSOC shared) biomarkers (e.g., biomarkers identified herein) are able to discriminate ovarian cancers from other type of cancers. This shows that the biomarkers are selective for ovarian cancers, or cancer of fallopian tube origin. The data shows that the biomarkers have the ability to discriminate ovarian cancer from 12 different types of other common types of cancers, such as leukemia, breast—invasive and DCIS, cervical, renal, colon, endometrial, brain, lung, lymphoma, pancreatic, and thyroid. The differentiation can be with these other cancer calls having AUCs comparable to healthy subjects versus the identified biomarkers recited herein for ovarian cancer that have much higher AUCs. The data shows that the ovarian cancer biomarkers described herein can be successful by being specific for ovarian cancer over other cancers.

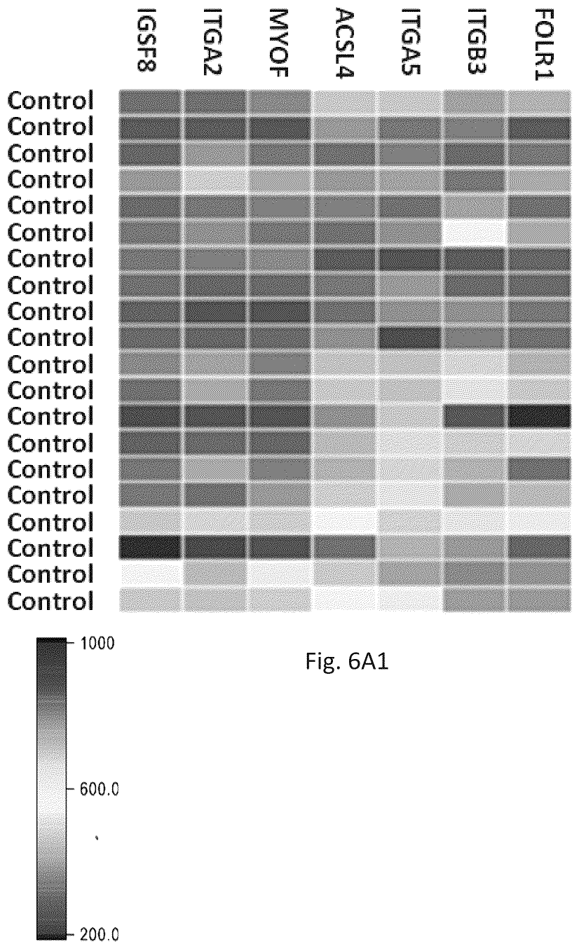

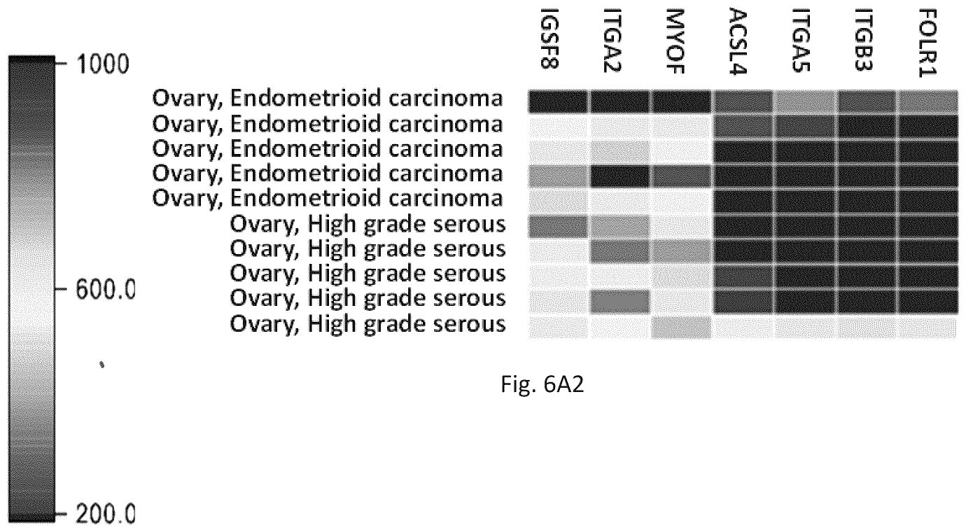

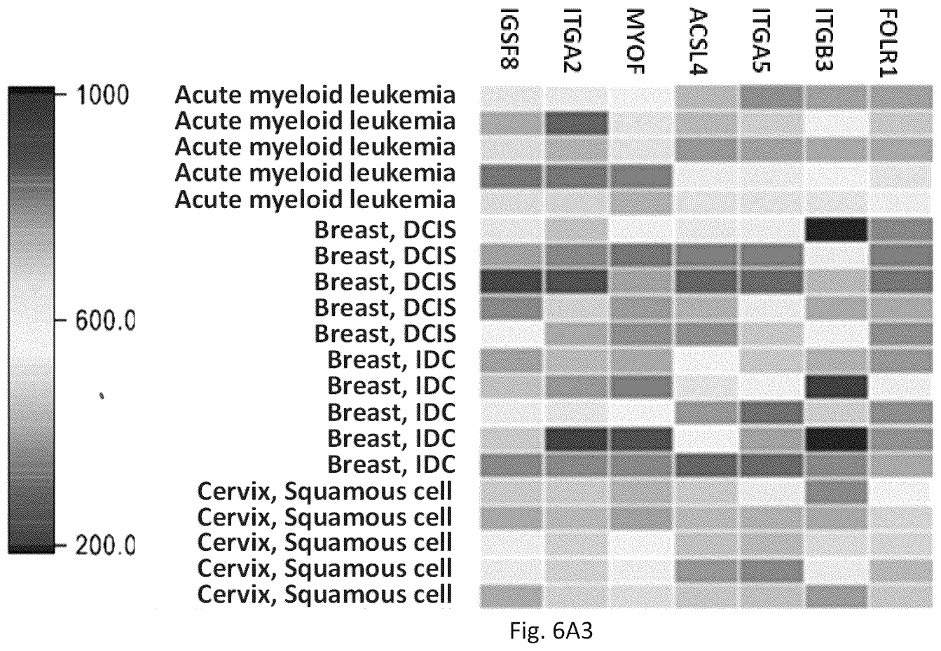

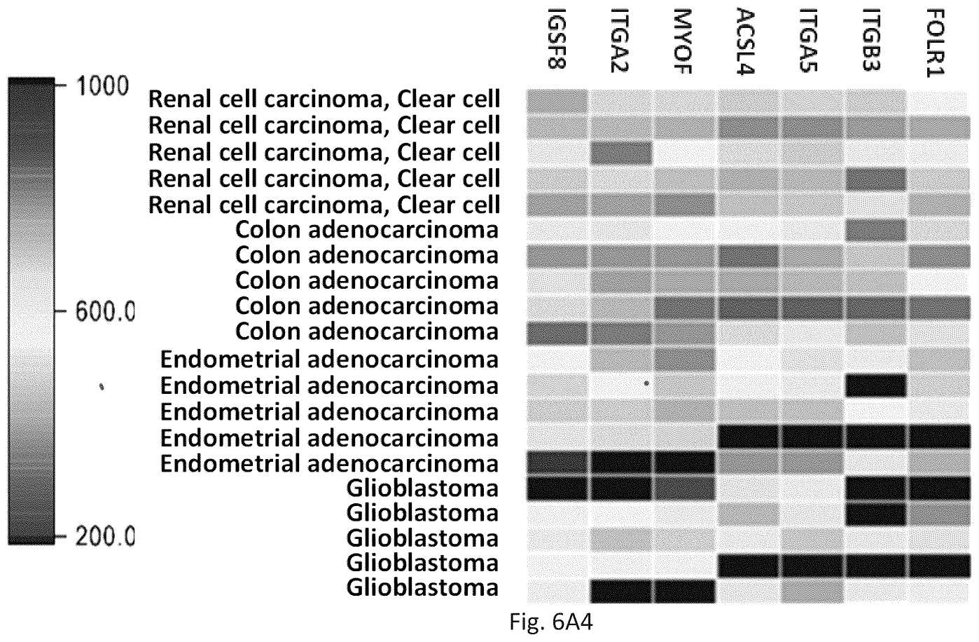

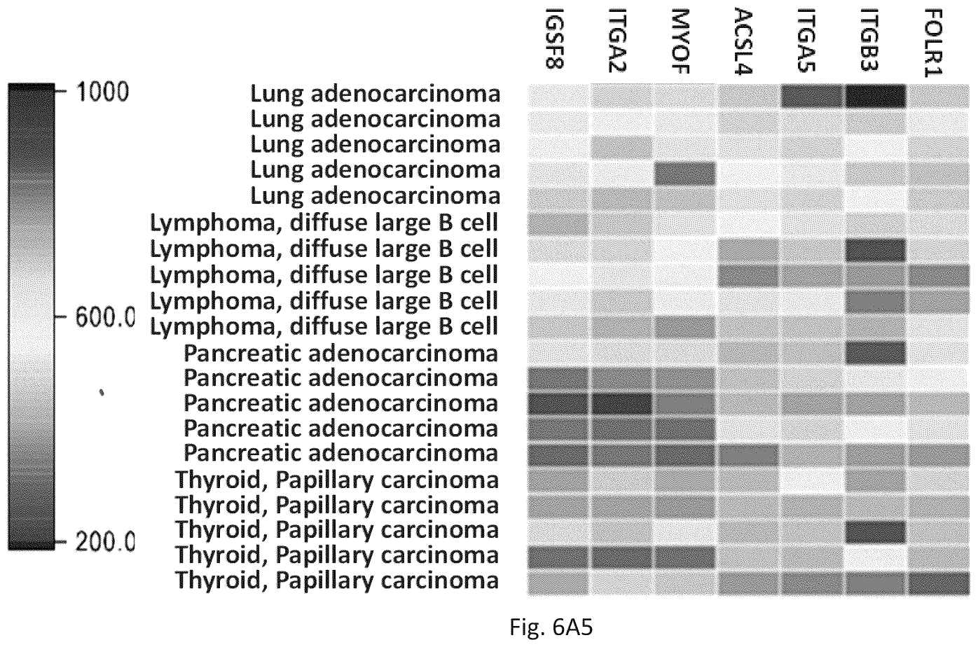

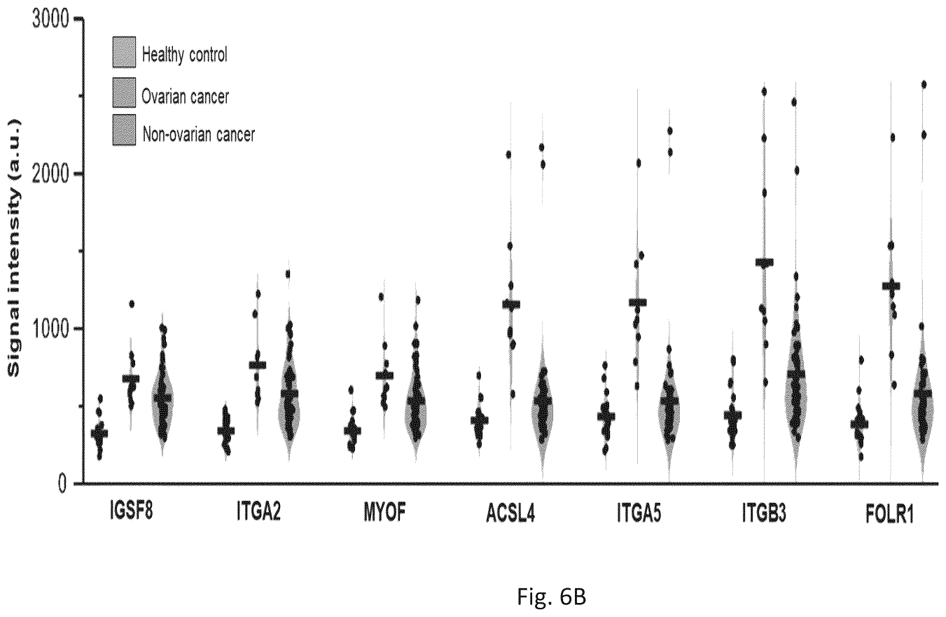

FIGS. 6A1-6A5 include heatmaps of the relative expression levels of the 7 exo-protein biomarkers in 70 cancer samples (10 ovarian cancer and 60 non-ovarian cancers) and 20 healthy controls. FIG. 6B is a scatter plot showing quantification of 6 transmembrane exo-protein biomarkers on captured CD81+ EVs for 70 cancer samples (10 ovarian cancer and 60 non-ovarian cancers) and 20 healthy controls. FOLR1 was included as a positive control for HGSOC.

A report can be prepared that identifies the treatment for the subject, and the report can be provided to the subject, or a medical professional, or a medical clinic, or a hospital or other treatment facility. Once the treatment is identified, the method can include an entity coordinating the performance of the treatment on the subject. Then, the subject can be treated as determined and reported. The report can be provided to the subject for consideration, whether in verbal or written or electronic form. The report can also include the recommendation for a treatment, and optionally treatment providers. Also, the report can include information about the outcomes of the treatments, including positive outcomes and/or negative outcomes. The patient can then be put on the waitlist, and can undergo further examinations.

To realize the above-mentioned report, a diagnosis results assessment system of the present invention includes: a diagnosis unit to perform a diagnosis of a subject to determine the presence of biomarkers, for generating diagnosis record information; a report storage unit to store a report describing a diagnosis result on the sample; a report analysis unit to analyze the diagnosis result described in the report stored in the report storage unit; and a report verification unit to compare the diagnosis result analyzed by the report analysis unit to the diagnosis record information, and determine a degree of matching on the diagnosis degree of the comparison result.

A diagnosis results assessment method of the present invention includes: a diagnosing step of performing a diagnosis of a sample to determine the presence or amount of biomarkers for generating diagnosis record information; a report storing step of storing a report describing the biomarkers and diagnosis result for the subject in a report storage unit; a report analyzing step of analyzing the diagnosis result described in the report stored in the report storage unit: and a report verifying step of comparing the diagnosis result analyzed in the report analyzing step to the diagnosis record information, and determining a degree of matching on the diagnosis degree of the comparison result.

A diagnosis results assessment device of the present invention includes: a diagnosis unit (e.g., computing system) to perform a pathological diagnosis of a sample from a subject for generating diagnosis record information; a report storage unit to store a report describing a pathological diagnosis result on the tissue specimen image: a report analysis unit to analyze the diagnosis result described in the report stored in the report storage unit; and a report verification unit to compare the diagnosis result analyzed by the report analysis unit to the diagnosis record information, and determine a degree of matching on the diagnosis degree of the comparison result.

Embodiments of the disclosure provide a computing system for generating a diagnosis report based on data from a panel of biological markers of a patient. The system includes a communication interface configured to receive the data acquired by a diagnostic data acquisition device. The system further includes at least one processor. The at least one processor is configured to detect a medical condition of the patient and parameters associated with the medical condition based on the diagnostic data. At least one processor is further configured to construct the diagnosis report based on the diagnostic data, wherein the diagnosis report includes at least information related to the biomarkers and the disease indication, and a description of the medical condition using the parameters. The system also includes a display configured to display the diagnosis report.

Embodiments of the disclosure also provide a method for generating a diagnosis report based on diagnostic data. The method includes receiving, by a communication interface, the diagnostic data that is acquired by performing the biomarker detection/quantification assay. The method further includes detecting, by at least one processor, a medical condition of the patient and parameters associated with the medical condition based on the diagnostic data. The method also includes constructing, by at least one processor, the diagnosis report based on the diagnostic data. The method additionally includes displaying the diagnosis report on a display, transmitting the report, printing the report on paper, or otherwise providing the report to the patient or medical entity.

Embodiments of the disclosure further provide a non-transitory computer-readable medium having instructions stored thereon that, when executed by one or more processors, causes one or more processors to perform a method for generating a diagnosis report based on diagnostic data of a patient. The method includes receiving the diagnostic data acquired by the performance of the diagnostic assays. The method further includes detecting a medical condition of the patient and parameters associated with the medical condition based on the diagnostic data. The method also includes constructing the diagnosis report based on the diagnostic data. The diagnosis report includes at least one data linking the patient to the diagnosed medical condition, and a description of the medical condition using the parameters. The method additionally includes displaying the diagnosis report.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.
ADDIN EN.REFLIST 1 Das, P. M. & Bast, R. C., Jr. Early detection of ovarian cancer. Biomark Med 2, 291-303, doi:10.2217/17520363.2.3.291 (2008).
2 Drescher, C. W. et al. Impact of screening test performance and cost on mortality reduction and cost-effectiveness of multimodal ovarian cancer screening. Cancer Prev Res (Phila) 5, 1015-1024, doi:10.1158/1940-6207.CAPR-11-0468 (2012).
3 van Nagell, J. R., Jr., Gallion, H. H., Pavlik, E. J. & DePriest, P. D. Ovarian cancer screening. Cancer 76, 2086-2091 (1995).
4 Siegel, R. L., Miller, K. D. & Jemal, A. Cancer statistics, 2015. CA Cancer J Clin 5-29, doi:10.3322/caac.21254 (2015).
5 Bowtell, D. D. The genesis and evolution of high-grade serous ovarian cancer. Nat Rev Cancer 10, 803-808, doi:10.1038/nrc2946 (2010).
6 Jelovac, D. & Armstrong, D. K. Recent progress in the diagnosis and treatment of ovarian cancer. CA Cancer J Clin 61, 183-203, doi:10.3322/caac.20113 (2011).
7 Kurman, R. J. & Shih Ie, M. Molecular pathogenesis and extraovarian origin of epithelial ovarian cancer—shifting the paradigm. Hum Pathol 42, 918-931, doi:10.1016/j.humpath.2011.03.003 (2011).
8 Badgwell, D. & Bast, R. C., Jr. Early detection of ovarian cancer. Dis Markers 23, 397-410 (2007).
9 Hoskins, W. J. Prospective on ovarian cancer: why prevent? J Cell Biochem Suppl 23, 189-199 (1995).
10 Lu, K. H. et al. A 2-stage ovarian cancer screening strategy using the Risk of Ovarian Cancer Algorithm (ROCA) identifies early-stage incident cancers and demonstrates high positive predictive value. Cancer 119, 3454-3461, doi:10.1002/cncr.28183 (2013).
11 Rein, B. J. et al. Potential markers for detection and monitoring of ovarian cancer. J Oncol 2011, 475983, doi:10.1155/2011/475983 (2011).
12 Bast, R. C., Jr. et al. Prevention and early detection of ovarian cancer: mission impossible? Recent Results Cancer Res 174, 91-100 (2007).
13 Skates, S. J. et al. Toward an optimal algorithm for ovarian cancer screening with longitudinal tumor markers. Cancer 76, 2004-2010 (1995).
14 Sevinc, A., Adli, M., Kalender, M. E. & Camci, C. Benign causes of increased serum CA-125 concentration. Lancet Oncol 8, 1054-1055, doi:10.1016/S1470-2045(07)70357-1 (2007).
Topalak, O. et al. Serum, pleural effusion, and ascites CA-125 levels in ovarian cancer and nonovarian benign and malignant diseases: a comparative study. Gynecol Oncol 108-113, doi:10.1006/gyno.2001.6575 (2002).
16 Jacobs, I. J. et al. Ovarian cancer screening and mortality in the UK Collaborative Trial of Ovarian Cancer Screening (UKCTOCS): a randomised controlled trial. Lancet 387, 945-956, doi:10.1016/S0140-6736(15)01224-6 (2016).
17 Menon, U. et al. Ovarian cancer population screening and mortality after long-term follow-up in the UK Collaborative Trial of Ovarian Cancer Screening (UKCTOCS): a randomised controlled trial. Lancet 397, 2182-2193, doi:10.1016/S0140-6736(21)00731-5 (2021).
18 Labidi-Galy, S. I. et al. High grade serous ovarian carcinomas originate in the fallopian tube. Nat Commun 8, 1093, doi:10.1038/s41467-017-00962-1 (2017).
19 Perets, R. et al. Transformation of the fallopian tube secretory epithelium leads to high-grade serous ovarian cancer in Brca;Tp53;Pten models. Cancer Cell 24, 751-765, doi:10.1016/j.ccr.2013.10.013 (2013).
20 Lee, Y. et al. A candidate precursor to serous carcinoma that originates in the distal fallopian tube. J Pathol 211, 26-35, doi:10.1002/path.2091 (2007).
21 Wu, R. C. et al. Genomic landscape and evolutionary trajectories of ovarian cancer precursor lesions. J Pathol 248, 41-50, doi:10.1002/path.5219 (2019).
22 Anggraeni, T. D., Al Fattah, A. N. & Surya, R. Prophylactic salpingectomy and ovarian cancer: An evidence-based analysis. South Asian J Cancer 7, 42-45, doi:10.4103/sajc.sajc_187_17 (2018).
23 Yoon, S. H., Kim, S. N., Shim, S. H., Kang, S. B. & Lee, S. J. Bilateral salpingectomy can reduce the risk of ovarian cancer in the general population: A meta-analysis. Eur J Cancer 55, 38-46, doi:10.1016/j.ejca.2015.12.003 (2016).
24 Hoshino, A. et al. Tumour exosome integrins determine organotropic metastasis. Nature 527, 329-335, doi:10.1038/nature15756 (2015).
25 Colombo, M., Raposo, G. & Thery, C. Biogenesis, secretion, and intercellular interactions of exosomes and other extracellular vesicles. Annu Rev Cell Dev Biol 30, 255-289, doi:10.1146/annurev-cellbio-101512-122326 (2014).
26 Yanez-Mo, M. et al. Biological properties of extracellular vesicles and their physiological functions. J Extracell Vesicles 4, 27066, doi:10.3402/jev.v4.27066 (2015).
27 Hoshino, A. et al. Extracellular Vesicle and Particle Biomarkers Define Multiple Human Cancers. Cell 182, 1044-1061 e1018, doi:10.1016/j.cell.2020.07.009 (2020).
28 He, M., Crow, J., Roth, M., Zeng, Y. & Godwin, A. K. Integrated immunoisolation and protein analysis of circulating exosomes using microfluidic technology. Lab Chip 14, 3773-3780, doi:10.1039/c4lc00662c (2014).
29 Zhao, Z., Yang, Y., Zeng, Y. & He, M. A microfluidic ExoSearch chip for multiplexed exosome detection towards blood-based ovarian cancer diagnosis. Lab Chip 16, 489-496, doi:10.1039/c5lc01117e (2016).
30 Zhang, P. et al. Ultrasensitive detection of circulating exosomes with a 3D-nanopatterned microfluidic chip. Nature Biomedical Engineering, doi: 10.1038/s41551-019-0356-9 (2019).
31 Im, H. et al. Label-free detection and molecular profiling of exosomes with a nano-plasmonic sensor. Nat Biotechnol 32, 490-495, doi:10.1038/nbt.2886 (2014).
32 Zhang, P., Thou, X. & Zeng, Y. Multiplexed immunophenotyping of circulating exosomes on nano-engineered ExoProfile chip towards early diagnosis of cancer. Chem Sci 5495-5504, doi:10.1039/c9sc00961b (2019).
33 Crescitelli, R., Lasser, C. & Lotvall, J. Isolation and characterization of extracellular vesicle subpopulations from tissues. Nat Protoc 16, 1548-1580, doi:10.1038/s41596-020-00466-1 (2021).
34 Crescitelli, R. et al. Subpopulations of extracellular vesicles from human metastatic melanoma tissue identified by quantitative proteomics after optimized isolation. J Extracell Vesicles 9, 1722433, doi:10.1080/20013078.2020.1722433 (2020).
35 Thery, C. et al. Minimal information for studies of extracellular vesicles 2018 (MISEV2018): a position statement of the International Society for Extracellular Vesicles and update of the MISEV2014 guidelines. J Extracell Vesicles 7, 1535750, doi:10.1080/20013078.2018.1535750 (2018).
36 Thippabhotla, S., Thong, C. & He, M. 3D cell culture stimulates the secretion of in vivo like extracellular vesicles. Sci Rep 9, 13012, doi:10.1038/s41598-019-49671-3 (2019).
37 Tominaga, N. et al. RPN2-mediated glycosylation of tetraspanin CD63 regulates breast cancer cell malignancy. Mol Cancer 13, 134, doi:10.1186/1476-4598-13-134 (2014).
38 Kugeratski, F. G. et al. Quantitative proteomics identifies the core proteome of exosomes with syntenin-1 as the highest abundant protein and a putative universal biomarker. Nat Cell Biol 23, 631-641, doi:10.1038/s41556-021-00693-y (2021).
39 Jacobs, I. et al. Prevalence screening for ovarian cancer in postmenopausal women by CA 125 measurement and ultrasonography. BMJ 306, 1030-1034, doi:10.1136/bmj.306.6884.1030 (1993).
40 Moore, R. G. et al. A novel multiple marker bioassay utilizing HE4 and CA125 for the prediction of ovarian cancer in patients with a pelvic mass. Gynecol Oncol 112, 40-46, doi: 10.1016/j.ygyno.2008.08.031 (2009).
41 Miller, R. W. et al. Performance of the American College of Obstetricians and Gynecologists' ovarian tumor referral guidelines with a multivariate index assay. Obstet Gynecol 117, 1298-1306, doi:10.1097/AOG.0b013e31821b1d80 (2011).
42 Bantis, L. E. et al. The length of the receiver operating characteristic curve and the two cutoff Youden index within a robust framework for discovery, evaluation, and cutoff estimation in biomarker studies involving improper receiver operating characteristic curves. Stat Med 40, 1767-1789, doi:10.1002/sim.8869 (2021).
43 Seguin, L., Desgrosellier, J. S., Weis, S. M. & Cheresh, D. A. Integrins and cancer: regulators of cancer stemness, metastasis, and drug resistance. Trends Cell Biol 25, 234-240, doi:10.1016/j.tcb.2014.12.006 (2015).
44 Singh, A. et al. Exosome-mediated Transfer of alphavbeta3 Integrin from Tumorigenic to Nontumorigenic Cells Promotes a Migratory Phenotype. Mol Cancer Res 14, 1136-1146, doi:10.1158/1541-7786.MCR-16-0058 (2016).
45 Toth, E. A. et al. Formation of a protein corona on the surface of extracellular vesicles in blood plasma. J Extracell Vesicles 10, e12140, doi:10.1002/jev2.12140 (2021).
46 Kalli, K. R. et al. Folate receptor alpha as a tumor target in epithelial ovarian cancer. Gynecol Oncol 108, 619-626, doi:10.1016/j.ygyno.2007.11.020 (2008).
47 Huang, M. J. et al. FOLR1 increases sensitivity to cisplatin treatment in ovarian cancer cells. J Ovarian Res 11, 15, doi:10.1186/s13048-018-0387-y (2018).
48 Zhang, P. et al. Ultrasensitive detection of circulating exosomes with a 3D-nanopatterned microfluidic chip. Nat Biomed Eng 3, 438-451, doi:10.1038/s41551-019-0356-9 (2019).
49 Funston, G., Abel, G., Crosbie, E. J., Hamilton, W. & Walter, F. M. Could Ovarian Cancer Prediction Models Improve the Triage of Symptomatic Women in Primary Care? A Modelling Study Using Routinely Collected Data. Cancers (Basel) 13, doi:10.3390/cancers13122886 (2021).
50 Liang, B. et al. Characterization and proteomic analysis of ovarian cancer-derived exosomes. J Proteomics 80, 171-182, doi:10.1016/j.jprot.2012.12.029 (2013).
51 Dorayappan, K. D. P. et al. A Microfluidic Chip Enables Isolation of Exosomes and Establishment of Their Protein Profiles and Associated Signaling Pathways in Ovarian Cancer. Cancer Res 79, 3503-3513, doi:10.1158/0008-5472.CAN-18-3538 (2019).
52 Zaborowski, M. P. et al. Methods for Systematic Identification of Membrane Proteins for Specific Capture of Cancer-Derived Extracellular Vesicles. Cell Rep 27, 255-268 e256, doi:10.1016/j.celrep.2019.03.003 (2019).
53 Ma, L. L., Liang, L., Zhou, D. & Wang, S. W. Tumor suppressor miR-424-5p abrogates ferroptosis in ovarian cancer through targeting ACSL4. Neoplasma 68, 165-173, doi:10.4149/neo_2020_200707 N705 (2021).
54 Sawada, K. et al. Loss of E-cadherin promotes ovarian cancer metastasis via alpha 5-integrin, which is a therapeutic target. Cancer Res 68, 2329-2339, doi:10.1158/0008-5472.CAN-07-5167 (2008).
55 Ye, W. et al. STX4 expression is associated with classification, clinical stage and lymphatic metastasis in ovarian cancer. Transl Cancer Res 8, 466-472, doi:10.21037/tcr.2019.02.11 (2019).
56 Huang, Y. L. et al. Site-specific N-glycosylation of integrin alpha2 mediates collagen-dependent cell survival. iScience 24, 103168, doi:10.1016/j.isci.2021.103168 (2021).
57 Ma, L. et al. Overexpressed ITGA2 contributes to paclitaxel resistance by ovarian cancer cells through the activation of the AKT/FoxO1 pathway. Aging (Albany NY) 12, 5336-5351, doi:10.18632/aging.102954 (2020).
58 Partheen, K. et al. External validation suggests Integrin beta 3 as prognostic biomarker in serous ovarian adenocarcinomas. BMC Cancer 9, 336, doi:10.1186/1471-2407-9-336 (2009).
59 Gao, Q. et al. Heterotypic CAF-tumor spheroids promote early peritoneal metastatis of ovarian cancer. J Exp Med 216, 688-703, doi:10.1084/jem.20180765 (2019).
60 Blomme, A. et al. Myoferlin is a novel exosomal protein and functional regulator of cancer-derived exosomes. Oncotarget 7, 83669-83683, doi:10.18632/oncotarget.13276 (2016).
61 Yurkovetsky, Z. R., Linkov, F. Y., D, E. M. & Lokshin, A. E. Multiple biomarker panels for early detection of ovarian cancer. Future Oncol 2, 733-741, doi:10.2217/14796694.2.6.733 (2006).
62 Ferraro, S. et al. Serum human epididymis protein 4 vs carbohydrate antigen 125 for ovarian cancer diagnosis: a systematic review. J Clin Pathol 66, 273-281, doi:10.1136/jclinpath-2012-201031 (2013).
63 Karst, A. M., Levanon, K. & Drapkin, R. Modeling high-grade serous ovarian carcinogenesis from the fallopian tube. Proc Natl Acad Sci USA 108, 7547-7552, doi:10.1073/pnas.1017300108 (2011).
64 Detre, S., Saclani Jotti, G. & Dowsett, M. A “quickscore” method for immunohistochemical semiquantitation: validation for oestrogen receptor in breast carcinomas. J Clin Pathol 48, 876-878, doi:10.1136/jcp.48.9.876 (1995).
65 Luan, J. et al. Ultrabright fluorescent nanoscale labels for the femtomolar detection of analytes with standard bioassays. Nat Biomed Eng 4, 518-530, doi:10.1038/s41551-020-0547-4 (2020).
66 Wang, Z. et al. Microneedle patch for the ultrasensitive quantification of protein biomarkers in interstitial fluid. Nat Biomed Eng 5, 64-76, doi:10.1038/s41551-020-00672-y (2021).
67 Nelson, J. W., Sklenar, J., Barnes, A. P. & Minnier, J. The START App: a web-based RNAseq analysis and visualization resource. Bioinformatics 33, 447-449, doi:10.1093/bioinformatics/btw624 (2017).
68 Choi, H., Kim, S., Fermin, D., Tsou, C. C. & Nesvizhskii, A. I. QPROT: Statistical method for testing differential expression using protein-level intensity data in label-free quantitative proteomics. J Proteomics 129, 121-126, doi:10.1016/j jprot.2015.07.036 (2015).
69 Akaike, H. A New Look at the Statistical Model Identification. IEEE Transactions on Automatic Control 19, doi:10.1109/TAC.1974.1100705 (1974).
70 Youden, W. J. Index for rating diagnostic tests. Cancer 3, 32-35, doi:10.1002/1097-0142(1950)3:1<32::aid-cncr2820030106>3.0.co;2-3 (1950).

1. A method of reporting a diagnoses of cancer in a subject, comprising:
obtaining a biological sample from the subject;
measuring presence or amount of a combination of biomarkers in the biological sample, wherein the combination of biomarkers is selected from ACSL4, IGSF8, ITGA2, ITGA5, ITGB3, and MYOF and optionally STX4 and/or optionally FOLR1, wherein the presence of the biomarkers in the sample indicates presence of cancer cells in the subject, wherein an increased amount of the biomarkers in the same indicates presence of cancer cells in the subject;
determining whether the presence or amount of the combination of biomarkers indicates the presence of cancer cells in the subject;
preparing a report on the presence of cancer cells in the subject, wherein the report includes an association of the combination of biomarkers and the presence of cancer cells; and
providing the report to the subject or to a medical entity or medical practitioner.

2. The method of claim 1, wherein the report includes one or more of:
an identification of the ACSL4, IGSF8, ITGA2, ITGA5, ITGB3, and MYOF and optionally STX4 and/or optionally FOLR1 as biomarkers of ovarian cancer;
a measurement data that indicates the presence of ACSL4, IGSF8, ITGA2, ITGA5, ITGB3, and MYOF and optionally STX4 and/or optionally FOLR1;
a measurement data that indicates the increased amount of ACSL4, IGSF8, ITGA2, ITGA5, ITGB3, and MYOF and optionally STX4 and/or optionally FOLR1;
a measurement data of a control for ACSL4, IGSF8, ITGA2, ITGA5, ITGB3, and MYOF and optionally STX4 and/or optionally FOLR1;
a statement of possible outcomes of ovarian cancer;
a statement of treatments for ovarian cancer, wherein the treatments are selected from chemotherapy, radiation therapy, surgical removal of the ovarian cancer, or combinations thereof;
a statement of possible outcomes of treatments for ovarian cancer; or
a listing of medical entities that perform, oversee, or control the treatments for ovarian cancer.

3. The method of claim 1, wherein the measuring and determining steps include reverse-transcription polymerase chain reaction (RT-PCR) or reverse-transcription quantitative polymerase chain reaction (RT-qPCR) to determine the presence or amount of the combination of biomarkers.

4. The method of claim 1, wherein the measuring and determining steps include:
using a binding agent to bind with a protein form of each of the biomarkers of the combination of biomarkers; and
detecting binding of the binding agent with the protein form of each of the biomarkers.

5. The method of claim 1, wherein the measuring and determining steps include:
using a binding agent to bind with a mRNA form of each of the biomarkers of the combination of biomarkers, wherein each binding agent is a nucleic acid that hybridizes with the mRNA form of the respective mRNA form of each of the biomarkers; and
detecting binding of the binding agent with the mRNA form of each of the biomarkers.

6. The method of claim 1, comprising:
detecting a panel of extracellular vesicle-associated protein biomarkers in a female human subject, comprising:
a) obtaining extracellular vesicle-associated proteins from a biological sample from the female human subject;
b) providing a panel of binding agents to the extracellular vesicle-associated proteins, wherein the binding agents are configured to bind with extracellular vesicle-associated proteins of ACSL4, IGSF8, ITGA2, ITGA5, ITGB3, and MYOF and optionally STX4 and/or optionally FOLR1;
c) assaying for binding of a plurality of the binding agents of the panel with the plurality of extracellular vesicle-associated proteins;
d) detecting binding of the plurality of binding agents of the panel with the plurality of extracellular vesicle-associated proteins, the detected plurality of extracellular vesicle-associated proteins being the extracellular vesicle-associated protein biomarkers;
e) preparing a report on the detection of binding of the binding agents to the extracellular vesicle-associated proteins of ACSL4, IGSF8, ITGA2, ITGA5, ITGB3, and MYOF and optionally STX4 and/or optionally FOLR1; and
f) providing the report to the subject or to a medical entity or medical practitioner.

7. The method of claim 6, wherein the binding agents are selected from antibodies, antibody fragments, aptamers, or combinations thereof.

8. The method of claim 6, wherein the panel of binding agents are selective for at least ACSL4, IGSF8, ITGA2, ITGA5, ITGB3, FOLR1, and MYOF based on higher area under curve (AUC) over a negative control.

9. The method of claim 6, wherein the panel of binding agents are selective for at least ACSL4, IGSF8, ITGA2, ITGA5, ITGB3, MYOF, FOLR1, and STX4 based on higher area under curve (AUC) over a negative control.

10. The method of claim 6, wherein the assaying is a protein binding assay where each binding agent binds with a respective extracellular vesicle-associated protein biomarker from the plurality of extracellular vesicle-associated proteins.

11. The method of claim 10, further comprising quantifying levels of the extracellular vesicle-associated protein biomarkers using fluorescence.

12. The method of claim 11, wherein levels of the extracellular vesicle-associated protein biomarkers are higher than control proteins.

13. The method of claim 6, further comprising selecting the female human subject by at least one of:
to be suspected of having ovarian cancer or is positive for BRCA1, BRCA2 mutation or other cancer susceptibility genes;
is in a risk group for developing ovarian cancer; or
is devoid of symptoms of ovarian cancer.

14. The method of claim 10, further comprising applying secondary antibodies conjugated to biotin to the binding agents and bound extracellular vesicle-associated protein biomarkers, which are used as fluorescent reporters.

15. The method of claim 6, wherein the report includes one or more of:
an identification of the ACSL4, IGSF8, ITGA2, ITGA5, ITGB3, and MYOF and optionally STX4 and/or optionally FOLR1 as biomarkers of ovarian cancer;
a measurement data that indicates the increased amount of ACSL4, IGSF8, ITGA2, ITGA5, ITGB3, and MYOF and optionally STX4 and/or optionally FOLR1;
a measurement data of a control for ACSL4, IGSF8, ITGA2, ITGA5, ITGB3, and MYOF and optionally STX4 and/or optionally FOLR1;
a statement of possible outcomes of ovarian cancer;
a statement of treatments for ovarian cancer, wherein the treatments are selected from chemotherapy, radiation therapy, surgical removal of the ovarian cancer, or combinations thereof;
a statement of possible outcomes of treatments for ovarian cancer; or
a listing of medical entities that perform, oversee, or control the treatments for ovarian cancer.

16. A panel of binding agents for extracellular vesicle-associated protein biomarkers of ovarian cancer comprising:
a plurality of binding agents that selectively bind to the extracellular vesicle-associated protein biomarkers, wherein the binding agents are configured to selectively bind with a plurality extracellular vesicle-associated protein biomarkers consisting of ACSL4, IGSF8, ITGA2, ITGA5, ITGB3, and MYOF, and optionally STX4 and/or optionally FOLR1.

17. An assay system comprising:
a biochip functionalized for antibody conjugation; and
at least one composition having the panel of binding agents of claim 16.

18. A method for treating a female human subject for ovarian cancer, comprising:
a) obtaining extracellular vesicle-associated proteins from a biological sample from the female human subject;
b) providing a panel of binding agents to the extracellular vesicle-associated proteins, wherein the binding agents are configured to bind with extracellular vesicle-associated proteins of ACSL4, IGSF8, ITGA2, ITGA5, ITGB3, and MYOF and optionally STX4 and/or optionally FOLR1;
c) assaying for binding of a plurality of the binding agents of the panel with the plurality of extracellular vesicle-associated proteins;
d) detecting binding of the plurality of binding agents of the panel with the plurality of extracellular vesicle-associated proteins, the detected plurality of extracellular vesicle-associated proteins being the extracellular vesicle-associated protein biomarkers;
e) preparing a report on the detection of binding of the binding agents to the extracellular vesicle-associated proteins of ACSL4, IGSF8, ITGA2, ITGA5, ITGB3, and MYOF and optionally STX4 and/or optionally FOLR1, wherein the report has an identification of the ACSL4, IGSF8, ITGA2, ITGA5, ITGB3, and MYOF and optionally STX4 and/or optionally FOLR1 as biomarkers of ovarian cancer; and
f) providing the subject to get a treatment for the ovarian cancer, wherein the subject undergoes the treatment for ovarian cancer.

19. The method of claim 18, comprising monitoring the patient during the performance of the treatment for ovarian cancer.

20. The method of claim 18, wherein the treatments for ovarian cancer are selected from chemotherapy, radiation therapy, surgical removal of the ovarian cancer, or combinations thereof.

21. The method of claim 20, comprising the subject undergoing the treatment of ovarian cancer.

22. The method of claim 18, further comprising selecting the female human subject by at least one of:
to be suspected of having ovarian cancer or is positive for BRCA1, BRCA2 mutation or other cancer susceptibility genes;
is in a risk group for developing ovarian cancer; or
is devoid of symptoms of ovarian cancer.

23. The method of claim 18, comprising after the subject undergoes the treatment for ovarian cancer, performing steps a)-d) on the subject, further comprising:
g) preparing a second report on the detection of binding of the binding agents to the extracellular vesicle-associated proteins of ACSL4, IGSF8, ITGA2, ITGA5, ITGB3, and MYOF and optionally STX4 and/or optionally FOLR1; and
h) providing the report to the subject or to a medical entity or medical practitioner.

24. The method of claim 23, comprising performing a subsequent treatment if the second report provides an indication of ovarian cancer in the subject based on the extracellular vesicle-associated proteins of ACSL4, IGSF8, ITGA2, ITGA5, ITGB3, and MYOF and optionally STX4 and/or optionally FOLR1.
